(19) United States
(12) Patent Application Publication
(10) Pub. No.: US 2022/0288223 A1
(43) Pub. Date: Sep. 15, 2022

(54) ACTIVATABLE SPECIFIC BINDING MEMBER COMPLEXES, AND METHODS OF MAKING AND USING SAME

(71) Applicant: The Regents of the University of California, Oakland, CA (US)

(72) Inventors: Michael A. Whitney, San Diego, CA (US); Stephen Adams, Poway, CA (US); Qing Xiong, San Diego, CA (US); Dina Hin-Gorani, San Diego, CA (US)

(21) Appl. No.: 17/438,364

(22) PCT Filed: Mar. 12, 2020
(86) PCT No.: PCT/US20/22451

§ 371 Date: Sep. 10, 2021

Related U.S. Application Data
(60) Provisional application No. 62/817,062, filed on Mar. 12, 2019.

(51) Int. Cl. A61K 47/68; A61K 47/60; C07K 16/28

(52) U.S. Cl. CPC A61K 47/6849; A61K 47/60; C07K 16/2863; C07K 2317/24

(57) ABSTRACT

Disclosed herein, the invention pertains to methods and compositions involving activatable specific binding member complex(es).

### STATEMENT OF FEDERALLY-SPONSORED RESEARCH

This invention was made with government support under grants EB014929, GM086197, and NS090590 awarded by The National Institutes of Health. The government has certain rights in the invention.

### FIELD OF THE INVENTION

The present invention relates generally to the fields of activatable specific binding member complexes, and methods of making and using same.

### BACKGROUND OF THE INVENTION

Antibody therapy is currently one of the most widely accepted and rapidly growing area for biotherapeutics. Monoclonal antibodies and antibody drug conjugates are clinically approved to treat cancer and proinflammatory diseases including arthritis, multiple sclerosis, psoriasis, colitis, asthma and osteoporosis. Although generally safe, many of these antibody therapies have significant side effects often caused by the antibody's reactivity at off target locations in normal tissue. For example, cetuximab is widely used to treat metastatic colon and advanced or recurrent heads and neck cancer despite having dose limiting skin toxicities which causes a severe skin rash in approximately 90% of patients. This toxicity can lead to dose modifications, patient non-compliance, termination of treatment, or a combination thereof.

“Probodys” have been reported, which are genetically encoded pro-antibodies that encode a masking domain attached to an antibody as a single polypeptide chain. These pro-antibodies are functionally activated by protease which cleaves an amino acid encoded protease substrate to release the un-masked antibody (1-3). These functional antibodies utilize genetic encoding which limit their flexibility to change avidity, linker properties or incorporate both small molecule and non-natural amino acids into linker, protease substrate and antibody binding (masking) domain.

There are other reports of protease activated antibodies (4) (5).

### SUMMARY OF THE INVENTION

The present invention recognizes that the current state of the art of activatable specific binding member complexes lack flexibility in their design and structure.

A first aspect of the present invention generally relates to an activatable specific binding member complex.

A second aspect of the present invention generally relates to a method of making an activatable specific binding member complex.

A third aspect of the present invention generally relates to a method of modifying an antibody or active fragment of an antibody.

A fourth aspect of the present invention generally relates to a method of using an activatable specific binding member complex.

### BRIEF DESCRIPTION OF THE DRAWINGS

FIG. 1 depicts one aspect of an activatable specific binding member complex structure and modification that occur during protease activation and release of the complementary binding member.

FIG. 2 depicts one aspect of a complementary binding member/linker with NHS-ester, PEG24 linker, thrombin cleavage substrate and complementary binding member that binds cetuximab.

FIG. 3 depicts one aspect of specific localization of protease activated cell penetrating peptides cleaved by MMPs, uPA, plasmin and cathepsins in an animal model of head and neck cancer.

FIG. 4 depicts one aspect of functional activation of activatable cetuximab complex resulting in selective binding to EGFR. PEG24-Nle-TPRSFL-Cetuximab complementary binding member was covalently linked to Cetuximab (PA-cetuximab). EGFR binding curve shows thrombin protease activation of PA-Cetuximab (dotted line versus blocked PA-cetuximab solid line). Thrombin cleavage has no detected effect on unmodified cetuximab (red lines). Immuhistocytochemistry in head and neck cancer cell line (Cal-27) shows binding with Cetuximab (B) and PA-cetuximab (D) (HRP-dependent DAB staining on membranes), thrombin activation blocks binding of PA-cetuximab (C) equivalent with no antibody control (E).

FIG. 5 depicts one aspect of physical modification (change in molecular weight) of thrombin activatable cetuximab complex by addition of thrombin using gel electrophoresis.

FIG. 6 depicts one aspect of the structure of a protease activatable antibody and modification that occur during protease activation that release the large sterically restricting inhibitor domain.

FIG. 7 depicts one aspect of physical modification (change in molecular weight) of functionally inactivated cetuximab by addition large steric polyethylene glycol inhibitory groups.

FIG. 8 depicts one aspect of functional inhibition of cetuximab with synthetically attached large sterically inhibitor polyethylene glycol groups.

### DETAILED DESCRIPTION OF THE INVENTION

### Certain Definitions

The following terms have the meanings ascribed to them unless specified otherwise.

The terms cell penetrating peptide (CPP), activatable cell penetrating peptide (ACPP), membrane translocating sequence (MTS) and protein transduction domain are used interchangeably. As used herein, the terms mean a peptide (polypeptide or protein) sequence that is able to translocate across the plasma membrane of a cell. In some embodiments, a CPP facilitates the translocation of an extracellular molecule across the plasma membrane of a cell. In some embodiments, the CPP translocates across the plasma membrane by direct penetration of the plasma membrane, endocytosis-mediated entry, or the formation of a transitory structure. In some embodiments the MTS is not transported across the membrane of a cell, but is employed in an ex vivo assay or application.

As used herein, the term “aptamer” refers to a DNA or RNA molecule that has been selected from random pools based on their ability to bind other molecules with high affinity specificity based on non-Watson and Crick interactions with the target molecule (see, e.g., Cox and Ellington, Bioorg. Med. Chem. 9:2525-2531 (2001); Lee et al., Nuc. Acids Res. 32:D95-D100 (2004)). In some embodiments, the aptamer binds nucleic acids, proteins, small organic compounds, vitamins, inorganic compounds, cells, and even entire organisms.

The terms “polypeptide,” “peptide” and “protein” and derivatives thereof as used herein, are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds. As used herein, the terms “peptide” refers to a polymer of amino acid residues typically ranging in length from 2 to about 50 residues. In certain embodiments the peptide ranges in length from about 2, 3, 4, 5, 7, 9, 10, or 11 residues to about 50, 45, 40, 45, 30, 25, 20, or 15 residues. In certain embodiments the peptide ranges in length from about 8, 9, 10, 11, or 12 residues to about 15, 20 or 25 residues. Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a β-ester, a thioamide, phosphonamide, carbamate, hydroxylate, and the like (see, e.g., Spatola, Chem. Biochem. Amino Acids and Proteins 7: 267-357 (1983)), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “amino acid” and derivatives thereof as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be either D amino acids or L amino acids. In peptide sequences throughout the specification, lower case letters indicate the D isomer of the amino acid (conversely, upper case letters indicate the L isomer of the amino acid).

Amino acids may be referred to herein by either their commonly known three letter symbols or by the one-letter symbols recommended by the IUPAC-IUB Biochemical Nomenclature Commission.

Nucleotides, likewise, may be referred to by their commonly accepted single-letter codes.

One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

As used herein, a “linker” is any molecule capable of binding (e.g., covalently) portion A and portion B of a MTS molecule disclosed herein. Linkers include, but are not limited to, straight or branched chain carbon linkers, heterocyclic carbon linkers, peptide linkers, and polyether linkers. For example, poly(ethylene glycol) linkers are available from Quanta Biodesign, Powell, Ohio. These linkers optionally have amide linkages, sulfhydryl linkages, or heterofunctional linkages.

As used herein, the term “label” refers to any molecule that facilitates the visualization and/or detection of a MTS molecule disclosed herein. In some embodiments, the label is a fluorescent moiety.

The term “carrier” means an inert molecule that increases (a) plasma half-life and (b) solubility. In some embodiments, a carrier increases plasma half-life and solubility by reducing glomerular filtration. In some embodiments, a carrier increases tumor uptake due to enhanced permeability and retention (EPR) of tumor vasculature.

The term “thrombin” means an enzyme (EC 3.4.21.5) that cleaves fibrinogen molecules into fibrin monomers. Thrombin, acting through its G-protein coupled receptor PAR-I, is a key player in a wide range of vascular and extravascular disease processes throughout the body, including cancer, cardiovascular diseases, acute kidney injury, and stroke. In certain instances, thrombin activity increases over the course of atherosclerotic plaque development. In some embodiments, thrombin activity is a biomarker for atherosclerotic plaque development.

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a human. None of the terms require or are limited to situation characterized by the supervision (e.g. constant or intermittent) of a health care worker (e.g. a doctor, a registered nurse, a nurse practitioner, a physician's assistant, an orderly, or a hospice worker).

As used herein, the term “medical professional” means any health care worker. By way of non-limiting example, the health care worker may be a doctor, a registered nurse, a nurse practitioner, a physician's assistant, an orderly, or a hospice worker.

The terms “administer,” “administering,” “administration,” and derivatives thereof as used herein, refer to the methods that may be used to enable delivery of agents or compositions to the desired site of biological action. These methods include, but are not limited to parenteral injection (e.g., intravenous, subcutaneous, intraperitoneal, intramuscular, intravascular, intrathecal, intravitreal, infusion, or local). Administration techniques that are optionally employed with the agents and methods described herein, include e.g., as discussed in Goodman and Gilman, The Pharmacological Basis of Therapeutics, current ed, Pergamon, and Remington's, Pharmaceutical Sciences (current edition), Mack Publishing Co, Easton, Pa.

The term “pharmaceutically acceptable” and derivatives thereof as used herein, refers to a material that does not abrogate the biological activity or properties of the agents described herein, and is relatively nontoxic (ie, the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “surgery” and derivatives thereof as used herein, refers to any methods for that may be used to manipulate, change, or cause an effect by a physical intervention. These methods include, but are not limited to open surgery, endoscopic surgery, laparoscopic surgery, minimally invasive surgery, and robotic surgery.

The terms “neoplasm” or “neoplasia” and derivatives thereof as used herein, include any non-normal or non-standard cellular growth. Neoplasms can include tumors and cancers of any variety of stages, from benign to metastatic. Neoplasms can be primary or metastatic growths and can occur anywhere in a subject. Neoplasms can include neoplasms of the lung, skin, lymph, brain, nerves, muscle, breast, prostate, testis, pancreases, liver, kidneys, stomach, muscle, bone and blood. Neoplasms can be solid and non-solid tumors.

The terms “sample” or “samples” and derivatives thereof as used herein, include any samples obtained from a subject with can be employed with the methods described herein. Samples can include but are not limited to urine, blood, lymph, tears, mucus, saliva, biopsy or other sample tissue samples. Sample can be frozen, refrigerated, previously frozen, and/or stored for minutes, hours, days, weeks, months, years. Sampling techniques, handling and storage are well known and any such techniques for obtaining samples for use with the present invention are contemplated.

The following symbols, where used, are used with the indicated meanings F1=fluorescein, aca=ahx=X=ammohexanoyl linker (—HN—(CH2)<rCO-)aminohexanoyl, C=L-cysteine, E=L-glutamate, R=L-arginme, D=L-aspartate, K=L-lysine, A=L-alanine, r=D-arginine, c=D-cysteine, e=D-glutamate, P=L-proline, L=L-leucine, G=glycine, V=valine, I=isoleucine, M=methionine, F==phenylalanine, Y=tyrosine, W=tryptophan, H=histidine, Q=glutamine, N=asparagine, S=serine, T=threonine, o is 5-amino-3-oxapentanoyl linker, and C(me) is S-methylcysteine.

Antibodies that find use in the present invention can take on a number of formats as described herein, including traditional antibodies as well as antibody derivatives, fragments and mimetics, described herein and depicted in the figures.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. In some embodiments, the present invention can be directed to antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype.

Thus, “isotype” as used herein is meant any of the subclasses of immunoglobulins defined by the chemical and antigenic characteristics of their constant regions. It should be understood that therapeutic antibodies can also comprise hybrids of isotypes and/or subclasses. For example, as shown in US Publication 2009/0163699, incorporated by reference, the present invention the use of human IgG1/G2 hybrids.

The hypervariable region generally encompasses amino acid residues from about amino acid residues 31-35 (LCDR1; “L” denotes light chain), 50-65 (LCDR2) and 95-102 (LCDR3) in the light chain variable region and around about 24-34 (HCDR1; “H” denotes heavy chain), 5-56 (HCDR2), and 105-117 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991)(see, also Kabat numbering in Table 1 below) and/or those residues forming a hypervariable loop (e.g. residues 24-34 (LCDR1), 5-65 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are also described in the application.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering is as below, see Lafranc et al., Dev. Comp. Immunol. 27(1):55-77 (2003):

TABLE 1

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)).

Another type of Ig domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. The light chain generally comprises two domains, the variable light domain (containing the light chain CDRs and together with the variable heavy domains forming the Fv region), and a constant light chain region (often referred to as CL or Cκ).

Another region of interest for additional substitutions, outlined below, is the Fc region.

TABLE 2
Fc region

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

Thus, the present invention provides different antibody domains. As described herein and known in the art, the heterodimeric antibodies of the invention comprise different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

Thus, the “Fc domain” includes the —CH2-CH3 domain, and optionally a hinge domain (—H-CH2-CH3). In the embodiments herein, when a scFv is attached to an Fc domain, it is the C-terminus of the scFv construct that is attached to all or part of the hinge of the Fc domain; for example, it is generally attached to the sequence EPKS which is the beginning of the hinge. The heavy chain comprises a variable heavy domain and a constant domain, which includes a CH1-optional hinge-Fc domain comprising a CH2-CH3. The light chain comprises a variable light chain and the light constant domain. A scFv comprises a variable heavy chain, an scFv linker, and a variable light domain. In most of the constructs and sequences outlined herein, the C-terminus of the variable heavy chain is attached to the N-terminus of the scFv linker, the C-terminus of which is attached to the N-terminus of a variable light chain (N-vh-linker-vl-C) although that can be switched (N-vl-linker-vh-C).

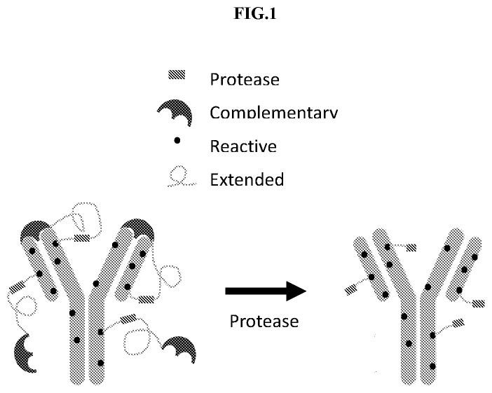

Some embodiments of the invention comprise at least one scFv domain, which, while not naturally occurring, generally includes a variable heavy domain and a variable light domain, linked together by a scFv linker. As outlined herein, while the scFv domain is generally from N- to C-terminus oriented as vh-scFv linker-vl, this can be reversed for any of the scFv domains (or those constructed using vh and vl sequences from Fabs), to vl-scFv linker-vh, with optional linkers at one or both ends depending on the format (see generally FIG. 1).

As shown herein, there are a number of suitable linkers (for use as either domain linkers or scFv linkers) that can be used to covalently attach the recited domains, including traditional peptide bonds, generated by recombinant techniques. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 37756), (GGGGS)n (SEQ ID NO: 37757), and (GGGS)n (SEQ ID NO: 37758), where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g. TCR, FcR, KIR), hinge region-derived sequences, and other natural sequences from other proteins.

In some embodiments, the linker is a “domain linker”, used to link any two domains as outlined herein together. For example, in FIG. 1F, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n (SEQ ID NO: 37756), (GGGGS)n (SEQ ID NO: 37757), and (GGGS)n (SEQ ID NO: 37758), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used.

In some embodiments, the linker is a scFv linker, used to covalently attach the vh and vl domains as discussed herein. Accordingly, the present invention further provides charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make triple F format heterodimeric antibody, the original pI of the Fv region for each of the desired antigen binding domains are calculated, and one is chosen to make an scFv, and depending on the pI, either positive or negative linkers are chosen.

The term “cetuximab”, sold as Erbitux®, as used herein refers to a recombinant, human/mouse chimeric monoclonal antibody that binds specifically to the extracellular domain of the human (EGFR). Cetuximab is composed of the Fv regions of a murine anti-EGFR antibody with human IgG1 heavy and kappa light chain constant regions and has an approximate molecular weight of 152 kDa. Cetuximab is produced in mammalian cell culture (murine myeloma). Erbitux is approved for the treatment of patients with metastatic colorectal cancer and whose tumor expresses EGFR. Cetuximab is described together with the respective method of preparation in, for example, U.S. Pat. No. 6,217,866.

The term “trastuzumab”, sold as Herceptin®, as used herein refers to a recombinant, humanized monoclonal antibody that binds specifically to the extracellular domain of the human (HER-2). Trastuzumab is composed of the Fv regions of humanized anti-HER-2 antibody with human IgG1 heavy and kappa light chain constant regions and has an approximate molecular weight of 145 kDa. Trastuzumab is produced in recombinant Chinese Hamster Ovary cells using a serum free medium. Trastuzumab is approved for the treatment of patients with early stage HER2-positive breast cancer, or metastatic breast cancer that substantially overexpress HER2. Trastuzumab is described together with the respective method of preparation in, for example, U.S. Pat. No. 6,870,034 B2.

The term “adalimumab”, sold as Humira®, as used herein refers to a fully human monoclonal antibody identified by phage display that binds specifically to the tumor necrosis factor alpha (TNFα). Adalimumab is composed of Fv regions that bind TNFα that were identified by phage display and human IgG1 heavy and kappa light chain constant regions and has an approximate molecular weight of 144 kDa. Adalimumab is produced in recombinant Chinese Hamster Ovary cells. Adalimumab is approved for the treatment rheumatoid arthritis, psoriatic arthritis, ankylosing spondylitis, Crohn's disease, ulcerative colitis, psoriasis, hidradenitis suppurativa, uveitis, and juvenile idiopathic arthritis. Adalimumab is described together with the respective method of preparation in, for example, U.S. Pat. No. 9,284,371 B2.

Unless defined otherwise, all technical and scientific terms used herein have the same meaning as commonly understood by one of ordinary skill in the art to which this invention belongs. Generally, the nomenclature used herein and the laboratory procedures in cell culture, chemistry, microbiology, molecular biology, cell science and cell culture described below are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well-known and commonly employed in the art. As employed throughout the disclosure, the following terms, unless otherwise indicated, shall be understood to have the following meanings:

For example:

“Directly” refers to direct causation of a process that does not require intermediate steps.

“Indirectly” refers to indirect causation that requires intermediate steps.

Other technical terms used herein have their ordinary meaning in the art that they are used, as exemplified by a variety of technical dictionaries.

The present invention recognizes that the current state of the art of activatable specific binding member complexes lack flexibility in their design and structure.

As a non-limiting introduction to the breath of the present invention, the present invention includes several general and useful aspects, including:
1) an activatable specific binding member complex;
2) a method of making an activatable specific binding member complex;
3) a method of modifying an antibody or active fragment of an antibody; and
4) a method of using an activatable specific binding member complex.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

### Activatable Specific Binding Member Complex

In an exemplary embodiment, the invention comprises an activatable specific binding member complex described herein. In an exemplary embodiment, the invention is an activatable specific binding member complex described herein. In an exemplary embodiment, the activatable specific binding member complex comprises a protease-activated antibody described herein. In an exemplary embodiment, the activatable specific binding member complex is a protease-activated antibody described herein.

In an exemplary embodiment, the activatable specific binding member complex, comprises:
a) at least one specific binding member, comprising:
   1) at least one specific binding region;
   2) at least one linker site that comprises a chemically reactive group; and
   3) at least one linker operably attached to at least one secondary site on said specific binding member by way of said linker site;
b) at least one complementary binding member, comprising:
   1) at least one mimetic binding domain;
      a. wherein said mimetic binding domain specifically binds and reversibly binds with said specific binding region; and
      b. wherein said mimetic binding domain has the same or different binding affinity for said specific binding region as that of the target binding domain for said specific binding region;
   2) at least one linker site that comprises a chemically reactive group;
   3) at least one linker operably attached to said complementary binding member by way of said linker site; and
c) at least one linker, comprising:
   1) at least one cleavable substrate;
   2) at least one extended linker region that comprises at least one non amino acid molecule; and
   3) wherein said linker is operably attached to said specific binding member and said complementary binding member to allow specific binding and reversible binding of said specific binding region with said mimetic binding domain so as to block the binding of said specific binding region with a moiety other than said binding mimetic domain;
wherein when said linker is cleaved at said cleavable substrate, said specific binding member and said complementary binding member become unbound by said linker and are capable of dissociating from each other;
further wherein when said specific binding member and said complimentary binding member become unbound, an activated specific binding member is formed; and
further wherein, said activated specific binding member functions such that said specific binding region can bind with at least one moiety other than said mimetic binding domain.

In an exemplary embodiment, the activatable specific binding member complex, comprises:
a) a first specific binding member, comprising:
   1) a first specific binding region, with binding affinity for a first target binding domain;
   2) a first linker site; and
b) a complementary binding member/linker according to the following formula:

R¹—R²—(R³)ₘ—R⁴—(R⁵)ₙ—R⁶
wherein R¹ is substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl; R² is a first complementary binding member; R³ is a first sublinker; m is either 0 or 1; R⁴ is a cleavable substrate; R⁵ is a second sublinker; n is either 0 or 1; R⁶ is the attachment point to the first linker site of the first specific binding member.

### a) Specific Binding Member:

### Cetuximab

In an exemplary embodiment, the first specific binding member comprises cetuximab. In an exemplary embodiment, the first specific binding member is derived from cetuximab. In an exemplary embodiment, the first specific binding member is cetuximab.

In an exemplary embodiment, the first specific binding member comprises a heavy chain variable region comprising SEQ ID NO:1. In an exemplary embodiment, the first specific binding member comprises a heavy chain variable region with at least 90%, or at least 92%, or at least 94%, or at least 96%, or at least 98%, or at least 99%, sequence identity with SEQ ID NO:1. In an exemplary embodiment, the first specific binding member is a heavy chain variable region comprising SEQ ID NO:1. In an exemplary embodiment, the first specific binding member is a heavy chain variable region with at least 90%, or at least 92%, or at least 94%, or at least 96%, or at least 98%, or at least 99%, sequence identity with SEQ ID NO:1.

In an exemplary embodiment, the first specific binding member comprises a light chain variable region comprising SEQ ID NO:2. In an exemplary embodiment, the first specific binding member comprises a light chain variable region with at least 90%, or at least 92%, or at least 94%, or at least 96%, or at least 98%, or at least 99%, sequence identity with SEQ ID NO:2. In an exemplary embodiment, the first specific binding member is a light chain variable region comprising SEQ ID NO:2. In an exemplary embodiment, the first specific binding member is a light chain variable region with at least 90%, or at least 92%, or at least 94%, or at least 96%, or at least 98%, or at least 99%, sequence identity with SEQ ID NO:2.

In an exemplary embodiment, the first specific binding member comprises a heavy chain variable region comprising SEQ ID NO:1 and a light chain variable region comprising SEQ ID NO:2. In an exemplary embodiment, the first specific binding member is a heavy chain variable region comprising SEQ ID NO:1 and a light chain variable region comprising SEQ ID NO:2.

In an exemplary embodiment, the first specific binding member comprises a heavy chain variable region comprising at least 90%, or at least 92%, or at least 94%, or at least 96%, or at least 98%, or at least 99%, sequence identity with SEQ ID NO:1 and a light chain variable region comprising at least 90%, or at least 92%, or at least 94%, or at least 96%, or at least 98%, or at least 99%, sequence identity with SEQ ID NO:2. In an exemplary embodiment, the first specific binding member is a heavy chain variable region with at least 90%, or at least 92%, or at least 94%, or at least 96%, or at least 98%, or at least 99%, sequence identity with SEQ ID NO:1 and a light chain variable region with at least 90%, or at least 92%, or at least 94%, or at least 96%, or at least 98%, or at least 99%, sequence identity with SEQ ID NO:2.

In an exemplary embodiment, the first specific binding member comprises a vhCDR1 comprising SEQ ID NO:3. In an exemplary embodiment, the first specific binding member comprises a vhCDR1 comprising at least 90%, or at least 92%, or at least 94%, or at least 96%, or at least 98%, or at least 99%, sequence identity with SEQ ID NO:3. In an exemplary embodiment, the first specific binding member is a vhCDR1 with SEQ ID NO:3. In an exemplary embodiment, the first specific binding member is a vhCDR1 with at least 90%, or at least 92%, or at least 94%, or at least 96%, or at least 98%, or at least 99%, sequence identity with SEQ ID NO:3.

In an exemplary embodiment, the first specific binding member comprises a vhCDR2 comprising SEQ ID NO:4. In an exemplary embodiment, the first specific binding member comprises a vhCDR2 comprising at least 90%, or at least 92%, or at least 94%, or at least 96%, or at least 98%, or at least 99%, sequence identity with SEQ ID NO:4. In an exemplary embodiment, the first specific binding member is a vhCDR2 with SEQ ID NO:4. In an exemplary embodiment, the first specific binding member is a vhCDR2 with at least 90%, or at least 92%, or at least 94%, or at least 96%, or at least 98%, or at least 99%, sequence identity with SEQ ID NO:4.

In an exemplary embodiment, the first specific binding member comprises a vhCDR3 comprising SEQ ID NO:5. In an exemplary embodiment, the first specific binding member comprises a vhCDR3 comprising at least 90%, or at least 92%, or at least 94%, or at least 96%, or at least 98%, or at least 99%, sequence identity with SEQ ID NO:5. In an exemplary embodiment, the first specific binding member is a vhCDR3 with SEQ ID NO:5. In an exemplary embodiment, the first specific binding member is a vhCDR3 with at least 90%, or at least 92%, or at least 94%, or at least 96%, or at least 98%, or at least 99%, sequence identity with SEQ ID NO:5.

In an exemplary embodiment, the first specific binding member comprises a vlCDR1 comprising SEQ ID NO:6. In an exemplary embodiment, the first specific binding member comprises a vlCDR1 comprising at least 90%, or at least 92%, or at least 94%, or at least 96%, or at least 98%, or at least 99%, sequence identity with SEQ ID NO:6. In an exemplary embodiment, the first specific binding member is a vlCDR1 with SEQ ID NO:6. In an exemplary embodiment, the first specific binding member is a vlCDR1 with at least 90%, or at least 92%, or at least 94%, or at least 96%, or at least 98%, or at least 99%, sequence identity with SEQ ID NO:6.

In an exemplary embodiment, the first specific binding member comprises a vlCDR2 comprising SEQ ID NO:7. In an exemplary embodiment, the first specific binding member comprises a vlCDR2 comprising at least 90%, or at least 92%, or at least 94%, or at least 96%, or at least 98%, or at least 99%, sequence identity with SEQ ID NO:7. In an exemplary embodiment, the first specific binding member is a vlCDR2 with SEQ ID NO:7. In an exemplary embodiment, the first specific binding member is a vlCDR2 with at least 90%, or at least 92%, or at least 94%, or at least 96%, or at least 98%, or at least 99%, sequence identity with SEQ ID NO:7.

In an exemplary embodiment, the first specific binding member comprises a vlCDR3 comprising SEQ ID NO:8. In an exemplary embodiment, the first specific binding member comprises a vlCDR3 comprising at least 90%, or at least 92%, or at least 94%, or at least 96%, or at least 98%, or at least 99%, sequence identity with SEQ ID NO:8. In an exemplary embodiment, the first specific binding member is a vlCDR3 with SEQ ID NO:8. In an exemplary embodiment, the first specific binding member is a vlCDR3 with at least 90%, or at least 92%, or at least 94%, or at least 96%, or at least 98%, or at least 99%, sequence identity with SEQ ID NO:8.

In an exemplary embodiment, the first specific binding member comprises a vhCDR1 comprising SEQ ID NO:3, a vhCDR2 comprising SEQ ID NO:4, a vhCDR3 comprising SEQ ID NO:5, a vlCDR1 comprising SEQ ID NO:6, a vlCDR2 comprising SEQ ID NO:7, and a vlCDR3 comprising SEQ ID NO:8. In an exemplary embodiment, the first specific binding member is a vhCDR1 comprising SEQ ID NO:3, a vhCDR2 comprising SEQ ID NO:4, a vhCDR3 comprising SEQ ID NO:5, a vlCDR1 comprising SEQ ID NO:6, a vlCDR2 comprising SEQ ID NO:7, and a vlCDR3 comprising SEQ ID NO:8.

In an exemplary embodiment, the first specific binding member comprises a vhCDR1 comprising at least 90%, or at least 92%, or at least 94%, or at least 96%, or at least 98%, or at least 99%, sequence identity with SEQ ID NO:3, a vhCDR2 comprising at least 90%, or at least 92%, or at least 94%, or at least 96%, or at least 98%, or at least 99%, sequence identity with SEQ ID NO:4, a vhCDR3 comprising at least 90%, or at least 92%, or at least 94%, or at least 96%, or at least 98%, or at least 99%, sequence identity with SEQ ID NO:5, a vlCDR1 comprising at least 90%, or at least 92%, or at least 94%, or at least 96%, or at least 98%, or at least 99%, sequence identity with SEQ ID NO:6, a vlCDR2 comprising at least 90%, or at least 92%, or at least 94%, or at least 96%, or at least 98%, or at least 99%, sequence identity with SEQ ID NO:7, and a vlCDR3 comprising at least 90%, or at least 92%, or at least 94%, or at least 96%, or at least 98%, or at least 99%, sequence identity with SEQ ID NO:8. In an exemplary embodiment, the first specific binding member is a vhCDR1 with at least 90%, or at least 92%, or at least 94%, or at least 96%, or at least 98%, or at least 99%, sequence identity with SEQ ID NO:3, a vhCDR2 with at least 90%, or at least 92%, or at least 94%, or at least 96%, or at least 98%, or at least 99%, sequence identity with SEQ ID NO:4, a vhCDR3 with at least 90%, or at least 92%, or at least 94%, or at least 96%, or at least 98%, or at least 99%, sequence identity with SEQ ID NO:5, a vlCDR1 with at least 90%, or at least 92%, or at least 94%, or at least 96%, or at least 98%, or at least 99%, sequence identity with SEQ ID NO:6, a vlCDR2 with at least 90%, or at least 92%, or at least 94%, or at least 96%, or at least 98%, or at least 99%, sequence identity with SEQ ID NO:7, and a vlCDR3 with at least 90%, or at least 92%, or at least 94%, or at least 96%, or at least 98%, or at least 99%, sequence identity with SEQ ID NO:8.

In an exemplary embodiment, the first specific binding member comprises a CDR of cetuximab. In an exemplary embodiment, the first specific binding member comprises between about 25% and about 99% of vhCDR1 cetuximab. In an exemplary embodiment, the first specific binding member comprises between about 25% and about 99% of vhCDR2 cetuximab. In an exemplary embodiment, the first specific binding member comprises between about 25% and about 99% of vhCDR3 cetuximab. In an exemplary embodiment, the first specific binding member comprises between about 25% and about 99% of vlCDR1 cetuximab. In an exemplary embodiment, the first specific binding member comprises between about 25% and about 99% of vlCDR2 cetuximab. In an exemplary embodiment, the first specific binding member comprises between about 25% and about 99% of vlCDR3 cetuximab.

In an exemplary embodiment, the first specific binding member comprises a CDR of cetuximab. In an exemplary embodiment, the first specific binding member comprises the vlCDR1, vlCDR2, vlCDR3, vhCDR1, vhCDR2 and vhCDR3 of cetuximab, wherein each CDR comprises 1, 2, 3, 4, 5, or 6 substitutions. In an exemplary embodiment, the first specific binding member comprises the vlCDR1, vlCDR2, vlCDR3, vhCDR1, vhCDR2 and vhCDR3 of cetuximab, wherein each CDR comprise no more than 6 substitutions. In an exemplary embodiment, the first specific binding member comprises the vlCDR1, vlCDR2, vlCDR3, vhCDR1, vhCDR2 and vhCDR3 of cetuximab, wherein each CDR comprise no more than 5 substitutions. In an exemplary embodiment, the first specific binding member comprises the vlCDR1, vlCDR2, vlCDR3, vhCDR1, vhCDR2 and vhCDR3 of cetuximab, wherein each CDR comprise no more than 4 substitutions. In an exemplary embodiment, the first specific binding member comprises the vlCDR1, vlCDR2, vlCDR3, vhCDR1, vhCDR2 and vhCDR3 of cetuximab, wherein each CDR comprise no more than 3 substitutions. In an exemplary embodiment, the first specific binding member comprises the vlCDR1, vlCDR2, vlCDR3, vhCDR1, vhCDR2 and vhCDR3 of cetuximab, wherein each CDR comprise no more than 2 substitutions. In an exemplary embodiment, the first specific binding member comprises the vlCDR1, vlCDR2, vlCDR3, vhCDR1, vhCDR2 and vhCDR3 of cetuximab, wherein each CDR comprise no more than 1 substitution. In an exemplary embodiment, the first specific binding member comprises the vlCDR1, vlCDR2, vlCDR3, vhCDR1, vhCDR2 and vhCDR3 of cetuximab. In an exemplary embodiment, the first specific binding member comprises at least the vlCDR3 and vhCDR3 of cetuximab. In an exemplary embodiment, the first specific binding member comprises at least the vlCDR3 and vhCDR3 of cetuximab, wherein each CDR comprises no more than 3 substitutions. In an exemplary embodiment, the first specific binding member comprises at least the vlCDR3 and vhCDR3 of cetuximab, wherein each CDR comprises no more than 2 substitutions. In an exemplary embodiment, the first specific binding member comprises at least the vlCDR3 and vhCDR3 of cetuximab, wherein each CDR comprises no more than 1 substitution.

### Trastuzumab

In an exemplary embodiment, the first specific binding member comprises trastuzumab. In an exemplary embodiment, the first specific binding member is derived from trastuzumab. In an exemplary embodiment, the first specific binding member is trastuzumab.

In an exemplary embodiment, the first specific binding member comprises a heavy chain variable region comprising SEQ ID NO:9. In an exemplary embodiment, the first specific binding member comprises a heavy chain variable region with at least 90%, or at least 92%, or at least 94%, or at least 96%, or at least 98%, or at least 99%, sequence identity with SEQ ID NO:9. In an exemplary embodiment, the first specific binding member is a heavy chain variable region comprising SEQ ID NO:9. In an exemplary embodiment, the first specific binding member is a heavy chain variable region with at least 90%, or at least 92%, or at least 94%, or at least 96%, or at least 98%, or at least 99%, sequence identity with SEQ ID NO:9.

In an exemplary embodiment, the first specific binding member comprises a light chain variable region comprising SEQ ID NO:10. In an exemplary embodiment, the first specific binding member comprises a light chain variable region with at least 90%, or at least 92%, or at least 94%, or at least 96%, or at least 98%, or at least 99%, sequence identity with SEQ ID NO:10. In an exemplary embodiment, the first specific binding member is a light chain variable region comprising SEQ ID NO:10. In an exemplary embodiment, the first specific binding member is a light chain variable region with at least 90%, or at least 92%, or at least 94%, or at least 96%, or at least 98%, or at least 99%, sequence identity with SEQ ID NO:10.

In an exemplary embodiment, the first specific binding member comprises a heavy chain variable region comprising SEQ ID NO:9 and a light chain variable region comprising SEQ ID NO:10. In an exemplary embodiment, the first specific binding member is a heavy chain variable region comprising SEQ ID NO:9 and a light chain variable region comprising SEQ ID NO:10.

In an exemplary embodiment, the first specific binding member comprises a heavy chain variable region comprising at least 90%, or at least 92%, or at least 94%, or at least 96%, or at least 98%, or at least 99%, sequence identity with SEQ ID NO:9 and a light chain variable region comprising at least 90%, or at least 92%, or at least 94%, or at least 96%, or at least 98%, or at least 99%, sequence identity with SEQ ID NO:10. In an exemplary embodiment, the first specific binding member is a heavy chain variable region with at least 90%, or at least 92%, or at least 94%, or at least 96%, or at least 98%, or at least 99%, sequence identity with SEQ ID NO:9 and a light chain variable region with at least 90%, or at least 92%, or at least 94%, or at least 96%, or at least 98%, or at least 99%, sequence identity with SEQ ID NO:10.

In an exemplary embodiment, the first specific binding member comprises a vhCDR1 comprising SEQ ID NO:11. In an exemplary embodiment, the first specific binding member comprises a vhCDR1 comprising at least 90%, or at least 92%, or at least 94%, or at least 96%, or at least 98%, or at least 99%, sequence identity with SEQ ID NO:11. In an exemplary embodiment, the first specific binding member is a vhCDR1 with SEQ ID NO:11. In an exemplary embodiment, the first specific binding member is a vhCDR1 with at least 90%, or at least 92%, or at least 94%, or at least 96%, or at least 98%, or at least 99%, sequence identity with SEQ ID NO:11.

In an exemplary embodiment, the first specific binding member comprises a vhCDR2 comprising SEQ ID NO:12. In an exemplary embodiment, the first specific binding member comprises a vhCDR2 comprising at least 90%, or at least 92%, or at least 94%, or at least 96%, or at least 98%, or at least 99%, sequence identity with SEQ ID NO:12. In an exemplary embodiment, the first specific binding member is a vhCDR2 with SEQ ID NO:12. In an exemplary embodiment, the first specific binding member is a vhCDR2 with at least 90%, or at least 92%, or at least 94%, or at least 96%, or at least 98%, or at least 99%, sequence identity with SEQ ID NO:12.

In an exemplary embodiment, the first specific binding member comprises a vhCDR3 comprising SEQ ID NO:13. In an exemplary embodiment, the first specific binding member comprises a vhCDR3 comprising at least 90%, or at least 92%, or at least 94%, or at least 96%, or at least 98%, or at least 99%, sequence identity with SEQ ID NO:13. In an exemplary embodiment, the first specific binding member is a vhCDR3 with SEQ ID NO:13. In an exemplary embodiment, the first specific binding member is a vhCDR3 with at least 90%, or at least 92%, or at least 94%, or at least 96%, or at least 98%, or at least 99%, sequence identity with SEQ ID NO:13.

In an exemplary embodiment, the first specific binding member comprises a vlCDR1 comprising SEQ ID NO:14. In an exemplary embodiment, the first specific binding member comprises a vlCDR1 comprising at least 90%, or at least 92%, or at least 94%, or at least 96%, or at least 98%, or at least 99%, sequence identity with SEQ ID NO:14. In an exemplary embodiment, the first specific binding member is a vlCDR1 with SEQ ID NO:14. In an exemplary embodiment, the first specific binding member is a vlCDR1 with at least 90%, or at least 92%, or at least 94%, or at least 96%, or at least 98%, or at least 99%, sequence identity with SEQ ID NO:14.

In an exemplary embodiment, the first specific binding member comprises a vlCDR2 comprising SEQ ID NO:15. In an exemplary embodiment, the first specific binding member comprises a vlCDR2 comprising at least 90%, or at least 92%, or at least 94%, or at least 96%, or at least 98%, or at least 99%, sequence identity with SEQ ID NO:15. In an exemplary embodiment, the first specific binding member is a vlCDR2 with SEQ ID NO:15. In an exemplary embodiment, the first specific binding member is a vlCDR2 with at least 90%, or at least 92%, or at least 94%, or at least 96%, or at least 98%, or at least 99%, sequence identity with SEQ ID NO:15.

In an exemplary embodiment, the first specific binding member comprises a vlCDR3 comprising SEQ ID NO:16. In an exemplary embodiment, the first specific binding member comprises a vlCDR3 comprising at least 90%, or at least 92%, or at least 94%, or at least 96%, or at least 98%, or at least 99%, sequence identity with SEQ ID NO:16. In an exemplary embodiment, the first specific binding member is a vlCDR3 with SEQ ID NO:16. In an exemplary embodiment, the first specific binding member is a vlCDR3 with at least 90%, or at least 92%, or at least 94%, or at least 96%, or at least 98%, or at least 99%, sequence identity with SEQ ID NO:16.

In an exemplary embodiment, the first specific binding member comprises a vhCDR1 comprising SEQ ID NO:11, a vhCDR2 comprising SEQ ID NO:12, a vhCDR3 comprising SEQ ID NO:13, a vlCDR1 comprising SEQ ID NO:14, a vlCDR2 comprising SEQ ID NO:15, and a vlCDR3 comprising SEQ ID NO:16. In an exemplary embodiment, the first specific binding member is a vhCDR1 comprising SEQ ID NO:11, a vhCDR2 comprising SEQ ID NO:12, a vhCDR3 comprising SEQ ID NO:13, a vlCDR1 comprising SEQ ID NO:14, a v1CDR2 comprising SEQ ID NO:15, and a vlCDR3 comprising SEQ ID NO:16.

In an exemplary embodiment, the first specific binding member comprises a vhCDR1 comprising at least 90%, or at least 92%, or at least 94%, or at least 96%, or at least 98%, or at least 99%, sequence identity with SEQ ID NO:11, a vhCDR2 comprising at least 90%, or at least 92%, or at least 94%, or at least 96%, or at least 98%, or at least 99%, sequence identity with SEQ ID NO:12, a vhCDR3 comprising at least 90%, or at least 92%, or at least 94%, or at least 96%, or at least 98%, or at least 99%, sequence identity with SEQ ID NO:13, a vlCDR1 comprising at least 90%, or at least 92%, or at least 94%, or at least 96%, or at least 98%, or at least 99%, sequence identity with SEQ ID NO:14, a vlCDR2 comprising at least 90%, or at least 92%, or at least 94%, or at least 96%, or at least 98%, or at least 99%, sequence identity with SEQ ID NO:15, and a vlCDR3 comprising at least 90%, or at least 92%, or at least 94%, or at least 96%, or at least 98%, or at least 99%, sequence identity with SEQ ID NO:16. In an exemplary embodiment, the first specific binding member is a vhCDR1 with at least 90%, or at least 92%, or at least 94%, or at least 96%, or at least 98%, or at least 99%, sequence identity with SEQ ID NO:11, a vhCDR2 with at least 90%, or at least 92%, or at least 94%, or at least 96%, or at least 98%, or at least 99%, sequence identity with SEQ ID NO:12, a vhCDR3 with at least 90%, or at least 92%, or at least 94%, or at least 96%, or at least 98%, or at least 99%, sequence identity with SEQ ID NO:13, a vlCDR1 with at least 90%, or at least 92%, or at least 94%, or at least 96%, or at least 98%, or at least 99%, sequence identity with SEQ ID NO:14, a vlCDR2 with at least 90%, or at least 92%, or at least 94%, or at least 96%, or at least 98%, or at least 99%, sequence identity with SEQ ID NO:15, and a vlCDR3 with at least 90%, or at least 92%, or at least 94%, or at least 96%, or at least 98%, or at least 99%, sequence identity with SEQ ID NO:16.

In an exemplary embodiment, the first specific binding member comprises a CDR of trastuzumab. In an exemplary embodiment, the first specific binding member comprises between about 25% and about 99% of vhCDR1 trastuzumab. In an exemplary embodiment, the first specific binding member comprises between about 25% and about 99% of vhCDR2 trastuzumab. In an exemplary embodiment, the first specific binding member comprises between about 25% and about 99% of vhCDR3 trastuzumab. In an exemplary embodiment, the first specific binding member comprises between about 25% and about 99% of vlCDR1 trastuzumab. In an exemplary embodiment, the first specific binding member comprises between about 25% and about 99% of v1CDR2 trastuzumab. In an exemplary embodiment, the first specific binding member comprises between about 25% and about 99% of vlCDR3 trastuzumab.

In an exemplary embodiment, the first specific binding member comprises a CDR of trastuzumab. In an exemplary embodiment, the first specific binding member comprises the vlCDR1, vlCDR2, vlCDR3, vhCDR1, vhCDR2 and vhCDR3 of trastuzumab, wherein each CDR comprises 1, 2, 3, 4, 5, or 6 substitutions. In an exemplary embodiment, the first specific binding member comprises the vlCDR1, vlCDR2, vlCDR3, vhCDR1, vhCDR2 and vhCDR3 of trastuzumab, wherein each CDR comprise no more than 6 substitutions. In an exemplary embodiment, the first specific binding member comprises the vlCDR1, vlCDR2, vlCDR3, vhCDR1, vhCDR2 and vhCDR3 of trastuzumab, wherein each CDR comprise no more than 5 substitutions. In an exemplary embodiment, the first specific binding member comprises the vlCDR1, vlCDR2, vlCDR3, vhCDR1, vhCDR2 and vhCDR3 of trastuzumab, wherein each CDR comprise no more than 4 substitutions. In an exemplary embodiment, the first specific binding member comprises the vlCDR1, vlCDR2, vlCDR3, vhCDR1, vhCDR2 and vhCDR3 of trastuzumab, wherein each CDR comprise no more than 3 substitutions. In an exemplary embodiment, the first specific binding member comprises the vlCDR1, vlCDR2, vlCDR3, vhCDR1, vhCDR2 and vhCDR3 of trastuzumab, wherein each CDR comprise no more than 2 substitutions. In an exemplary embodiment, the first specific binding member comprises the vlCDR1, vlCDR2, vlCDR3, vhCDR1, vhCDR2 and vhCDR3 of trastuzumab, wherein each CDR comprise no more than 1 substitution. In an exemplary embodiment, the first specific binding member comprises the vlCDR1, vlCDR2, vlCDR3, vhCDR1, vhCDR2 and vhCDR3 of trastuzumab. In an exemplary embodiment, the first specific binding member comprises at least the vlCDR3 and vhCDR3 of trastuzumab. In an exemplary embodiment, the first specific binding member comprises at least the vlCDR3 and vhCDR3 of trastuzumab, wherein each CDR comprises no more than 3 substitutions. In an exemplary embodiment, the first specific binding member comprises at least the vlCDR3 and vhCDR3 of trastuzumab, wherein each CDR comprises no more than 2 substitutions. In an exemplary embodiment, the first specific binding member comprises at least the vlCDR3 and vhCDR3 of trastuzumab, wherein each CDR comprises no more than 1 substitution.

### Adalimumab

In an exemplary embodiment, the first specific binding member comprises adalimumab. In an exemplary embodiment, the first specific binding member is derived from adalimumab. In an exemplary embodiment, the first specific binding member is adalimumab.

In an exemplary embodiment, the first specific binding member comprises a heavy chain variable region comprising SEQ ID NO:17. In an exemplary embodiment, the first specific binding member comprises a heavy chain variable region with at least 90%, or at least 92%, or at least 94%, or at least 96%, or at least 98%, or at least 99%, sequence identity with SEQ ID NO:17. In an exemplary embodiment, the first specific binding member is a heavy chain variable region comprising SEQ ID NO:17. In an exemplary embodiment, the first specific binding member is a heavy chain variable region with at least 90%, or at least 92%, or at least 94%, or at least 96%, or at least 98%, or at least 99%, sequence identity with SEQ ID NO:17.

In an exemplary embodiment, the first specific binding member comprises a light chain variable region comprising SEQ ID NO:18. In an exemplary embodiment, the first specific binding member comprises a light chain variable region with at least 90%, or at least 92%, or at least 94%, or at least 96%, or at least 98%, or at least 99%, sequence identity with SEQ ID NO:18. In an exemplary embodiment, the first specific binding member is a light chain variable region comprising SEQ ID NO:18. In an exemplary embodiment, the first specific binding member is a light chain variable region with at least 90%, or at least 92%, or at least 94%, or at least 96%, or at least 98%, or at least 99%, sequence identity with SEQ ID NO:18.

In an exemplary embodiment, the first specific binding member comprises a heavy chain variable region comprising SEQ ID NO:17 and a light chain variable region comprising SEQ ID NO:18. In an exemplary embodiment, the first specific binding member is a heavy chain variable region comprising SEQ ID NO:17 and a light chain variable region comprising SEQ ID NO:18.

In an exemplary embodiment, the first specific binding member comprises a heavy chain variable region comprising at least 90%, or at least 92%, or at least 94%, or at least 96%, or at least 98%, or at least 99%, sequence identity with SEQ ID NO:17 and a light chain variable region comprising at least 90%, or at least 92%, or at least 94%, or at least 96%, or at least 98%, or at least 99%, sequence identity with SEQ ID NO:18. In an exemplary embodiment, the first specific binding member is a heavy chain variable region with at least 90%, or at least 92%, or at least 94%, or at least 96%, or at least 98%, or at least 99%, sequence identity with SEQ ID NO:17 and a light chain variable region with at least 90%, or at least 92%, or at least 94%, or at least 96%, or at least 98%, or at least 99%, sequence identity with SEQ ID NO:18.

In an exemplary embodiment, the first specific binding member comprises a vhCDR1 comprising SEQ ID NO:19. In an exemplary embodiment, the first specific binding member comprises a vhCDR1 comprising at least 90%, or at least 92%, or at least 94%, or at least 96%, or at least 98%, or at least 99%, sequence identity with SEQ ID NO:19. In an exemplary embodiment, the first specific binding member is a vhCDR1 with SEQ ID NO:19. In an exemplary embodiment, the first specific binding member is a vhCDR1 with at least 90%, or at least 92%, or at least 94%, or at least 96%, or at least 98%, or at least 99%, sequence identity with SEQ ID NO:19.

In an exemplary embodiment, the first specific binding member comprises a vhCDR2 comprising SEQ ID NO:20. In an exemplary embodiment, the first specific binding member comprises a vhCDR2 comprising at least 90%, or at least 92%, or at least 94%, or at least 96%, or at least 98%, or at least 99%, sequence identity with SEQ ID NO:20. In an exemplary embodiment, the first specific binding member is a vhCDR2 with SEQ ID NO:20. In an exemplary embodiment, the first specific binding member is a vhCDR2 with at least 90%, or at least 92%, or at least 94%, or at least 96%, or at least 98%, or at least 99%, sequence identity with SEQ ID NO:20.

In an exemplary embodiment, the first specific binding member comprises a vhCDR3 comprising SEQ ID NO:21. In an exemplary embodiment, the first specific binding member comprises a vhCDR3 comprising at least 90%, or at least 92%, or at least 94%, or at least 96%, or at least 98%, or at least 99%, sequence identity with SEQ ID NO:21. In an exemplary embodiment, the first specific binding member is a vhCDR3 with SEQ ID NO:21. In an exemplary embodiment, the first specific binding member is a vhCDR3 with at least 90%, or at least 92%, or at least 94%, or at least 96%, or at least 98%, or at least 99%, sequence identity with SEQ ID NO:21.

In an exemplary embodiment, the first specific binding member comprises a vlCDR1 comprising SEQ ID NO:22. In an exemplary embodiment, the first specific binding member comprises a vlCDR1 comprising at least 90%, or at least 92%, or at least 94%, or at least 96%, or at least 98%, or at least 99%, sequence identity with SEQ ID NO:22. In an exemplary embodiment, the first specific binding member is a vlCDR1 with SEQ ID NO:22. In an exemplary embodiment, the first specific binding member is a vlCDR1 with at least 90%, or at least 92%, or at least 94%, or at least 96%, or at least 98%, or at least 99%, sequence identity with SEQ ID NO:22.

In an exemplary embodiment, the first specific binding member comprises a vlCDR2 comprising SEQ ID NO:23. In an exemplary embodiment, the first specific binding member comprises a vlCDR2 comprising at least 90%, or at least 92%, or at least 94%, or at least 96%, or at least 98%, or at least 99%, sequence identity with SEQ ID NO:23. In an exemplary embodiment, the first specific binding member is a vlCDR2 with SEQ ID NO:23. In an exemplary embodiment, the first specific binding member is a vlCDR2 with at least 90%, or at least 92%, or at least 94%, or at least 96%, or at least 98%, or at least 99%, sequence identity with SEQ ID NO:23.

In an exemplary embodiment, the first specific binding member comprises a vlCDR3 comprising SEQ ID NO:24. In an exemplary embodiment, the first specific binding member comprises a vlCDR3 comprising at least 90%, or at least 92%, or at least 94%, or at least 96%, or at least 98%, or at least 99%, sequence identity with SEQ ID NO:24. In an exemplary embodiment, the first specific binding member is a vlCDR3 with SEQ ID NO:24. In an exemplary embodiment, the first specific binding member is a vlCDR3 with at least 90%, or at least 92%, or at least 94%, or at least 96%, or at least 98%, or at least 99%, sequence identity with SEQ ID NO:24.

In an exemplary embodiment, the first specific binding member comprises a vhCDR1 comprising SEQ ID NO:19, a vhCDR2 comprising SEQ ID NO:20, a vhCDR3 comprising SEQ ID NO:21, a vlCDR1 comprising SEQ ID NO:22, a vlCDR2 comprising SEQ ID NO:23, and a vlCDR3 comprising SEQ ID NO:24. In an exemplary embodiment, the first specific binding member is a vhCDR1 comprising SEQ ID NO:19, a vhCDR2 comprising SEQ ID NO:20, a vhCDR3 comprising SEQ ID NO:21, a vlCDR1 comprising SEQ ID NO:22, a vlCDR2 comprising SEQ ID NO:23, and a vlCDR3 comprising SEQ ID NO:24.

In an exemplary embodiment, the first specific binding member comprises a vhCDR1 comprising at least 90%, or at least 92%, or at least 94%, or at least 96%, or at least 98%, or at least 99%, sequence identity with SEQ ID NO:19, a vhCDR2 comprising at least 90%, or at least 92%, or at least 94%, or at least 96%, or at least 98%, or at least 99%, sequence identity with SEQ ID NO:20, a vhCDR3 comprising at least 90%, or at least 92%, or at least 94%, or at least 96%, or at least 98%, or at least 99%, sequence identity with SEQ ID NO:21, a vlCDR1 comprising at least 90%, or at least 92%, or at least 94%, or at least 96%, or at least 98%, or at least 99%, sequence identity with SEQ ID NO:22, a vlCDR2 comprising at least 90%, or at least 92%, or at least 94%, or at least 96%, or at least 98%, or at least 99%, sequence identity with SEQ ID NO:23, and a vlCDR3 comprising at least 90%, or at least 92%, or at least 94%, or at least 96%, or at least 98%, or at least 99%, sequence identity with SEQ ID NO:24. In an exemplary embodiment, the first specific binding member is a vhCDR1 with at least 90%, or at least 92%, or at least 94%, or at least 96%, or at least 98%, or at least 99%, sequence identity with SEQ ID NO:19, a vhCDR2 with at least 90%, or at least 92%, or at least 94%, or at least 96%, or at least 98%, or at least 99%, sequence identity with SEQ ID NO:20, a vhCDR3 with at least 90%, or at least 92%, or at least 94%, or at least 96%, or at least 98%, or at least 99%, sequence identity with SEQ ID NO:21, a vlCDR1 with at least 90%, or at least 92%, or at least 94%, or at least 96%, or at least 98%, or at least 99%, sequence identity with SEQ ID NO:22, a vlCDR2 with at least 90%, or at least 92%, or at least 94%, or at least 96%, or at least 98%, or at least 99%, sequence identity with SEQ ID NO:23, and a vlCDR3 with at least 90%, or at least 92%, or at least 94%, or at least 96%, or at least 98%, or at least 99%, sequence identity with SEQ ID NO:24.

In an exemplary embodiment, the first specific binding member comprises a CDR of adalimumab. In an exemplary embodiment, the first specific binding member comprises between about 25% and about 99% of vhCDR1 adalimumab. In an exemplary embodiment, the first specific binding member comprises between about 25% and about 99% of vhCDR2 adalimumab. In an exemplary embodiment, the first specific binding member comprises between about 25% and about 99% of vhCDR3 adalimumab. In an exemplary embodiment, the first specific binding member comprises between about 25% and about 99% of vlCDR1 adalimumab. In an exemplary embodiment, the first specific binding member comprises between about 25% and about 99% of v1CDR2 adalimumab. In an exemplary embodiment, the first specific binding member comprises between about 25% and about 99% of vlCDR3 adalimumab.

In an exemplary embodiment, the first specific binding member comprises a CDR of adalimumab. In an exemplary embodiment, the first specific binding member comprises the vlCDR1, vlCDR2, vlCDR3, vhCDR1, vhCDR2 and vhCDR3 of adalimumab, wherein each CDR comprises 1, 2, 3, 4, 5, or 6 substitutions. In an exemplary embodiment, the first specific binding member comprises the vlCDR1, vlCDR2, vlCDR3, vhCDR1, vhCDR2 and vhCDR3 of adalimumab, wherein each CDR comprise no more than 6 substitutions. In an exemplary embodiment, the first specific binding member comprises the vlCDR1, vlCDR2, vlCDR3, vhCDR1, vhCDR2 and vhCDR3 of adalimumab, wherein each CDR comprise no more than 5 substitutions. In an exemplary embodiment, the first specific binding member comprises the vlCDR1, vlCDR2, vlCDR3, vhCDR1, vhCDR2 and vhCDR3 of adalimumab, wherein each CDR comprise no more than 4 substitutions. In an exemplary embodiment, the first specific binding member comprises the vlCDR1, vlCDR2, vlCDR3, vhCDR1, vhCDR2 and vhCDR3 of adalimumab, wherein each CDR comprise no more than 3 substitutions. In an exemplary embodiment, the first specific binding member comprises the vlCDR1, vlCDR2, vlCDR3, vhCDR1, vhCDR2 and vhCDR3 of adalimumab, wherein each CDR comprise no more than 2 substitutions. In an exemplary embodiment, the first specific binding member comprises the vlCDR1, vlCDR2, vlCDR3, vhCDR1, vhCDR2 and vhCDR3 of adalimumab, wherein each CDR comprise no more than 1 substitution. In an exemplary embodiment, the first specific binding member comprises the vlCDR1, vlCDR2, vlCDR3, vhCDR1, vhCDR2 and vhCDR3 of adalimumab. In an exemplary embodiment, the first specific binding member comprises at least the vlCDR3 and vhCDR3 of adalimumab. In an exemplary embodiment, the first specific binding member comprises at least the vlCDR3 and vhCDR3 of adalimumab, wherein each CDR comprises no more than 3 substitutions. In an exemplary embodiment, the first specific binding member comprises at least the vlCDR3 and vhCDR3 of adalimumab, wherein each CDR comprises no more than 2 substitutions. In an exemplary embodiment, the first specific binding member comprises at least the vlCDR3 and vhCDR3 of adalimumab, wherein each CDR comprises no more than 1 substitution.

### a1) Specific Binding Region:

In an exemplary embodiment, the first specific binding region comprises a CDR of cetuximab. In an exemplary embodiment, the first specific binding region comprises between about 25% and about 99% of vhCDR1 cetuximab. In an exemplary embodiment, the first specific binding region comprises between about 25% and about 99% of vhCDR2 cetuximab. In an exemplary embodiment, the first specific binding region comprises between about 25% and about 99% of vhCDR3 cetuximab. In an exemplary embodiment, the first specific binding region comprises between about 25% and about 99% of vlCDR1 cetuximab. In an exemplary embodiment, the first specific binding region comprises between about 25% and about 99% of vlCDR2 cetuximab. In an exemplary embodiment, the first specific binding region comprises between about 25% and about 99% of vlCDR3 cetuximab.

In an exemplary embodiment, the first specific binding region comprises a CDR of trastuzumab. In an exemplary embodiment, the first specific binding region comprises between about 25% and about 99% of vhCDR1 trastuzumab. In an exemplary embodiment, the first specific binding region comprises between about 25% and about 99% of vhCDR2 trastuzumab. In an exemplary embodiment, the first specific binding region comprises between about 25% and about 99% of vhCDR3 trastuzumab. In an exemplary embodiment, the first specific binding region comprises between about 25% and about 99% of vlCDR1 trastuzumab. In an exemplary embodiment, the first specific binding region comprises between about 25% and about 99% of vlCDR2 trastuzumab. In an exemplary embodiment, the first specific binding region comprises between about 25% and about 99% of vlCDR3 trastuzumab.

In an exemplary embodiment, the first specific binding region comprises a CDR of adalimumab. In an exemplary embodiment, the first specific binding region comprises between about 25% and about 99% of vhCDR1 adalimumab. In an exemplary embodiment, first specific binding region comprises between about 25% and about 99% of vhCDR2 adalimumab. In an exemplary embodiment, the first specific binding region comprises between about 25% and about 99% of vhCDR3 adalimumab. In an exemplary embodiment, the first specific binding region comprises between about 25% and about 99% of vlCDR1 adalimumab. In an exemplary embodiment, the first specific binding region comprises between about 25% and about 99% of vlCDR2 adalimumab. In an exemplary embodiment, the first specific binding region comprises between about 25% and about 99% of vlCDR3 adalimumab.

### a2) Linker Site:

In an exemplary embodiment, the first linker site is a lysine. In an exemplary embodiment, the first linker site is a cysteine. In an exemplary embodiment, the first linker site is a glutamine. In an exemplary embodiment, the first linker site is a non-native amino acid that has a reactive side chain which comprises ketone, azide, alkyne, alkene, and/or tetrazine.

### b1) Complementary Binding Member/Linker:

In an exemplary embodiment, the complementary binding member/linker is according to the following formula:

R¹—R²—(R³)ₘ—R⁴—(R⁵)ₙ—R⁶

wherein R¹ is a substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl; R² is a complementary binding member; R³ is a first sublinker; m is either 1 or 0; R⁴ is a cleavable substrate; R⁵ is a second sublinker; n is either 1 or 0; R⁶ is the attachment point to the specific binding member.

In an exemplary embodiment, R¹ is substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl. In an exemplary embodiment, R¹ comprises a dye, such as Cy5 or fluorescein amidite (FAM). In an exemplary embodiment, R¹ is substituted C₁-C₆ alkyl carbonyl. In an exemplary embodiment, R¹ is unsubstituted C₁-C₆ alkyl carbonyl. In an exemplary embodiment, R¹ is unsubstituted C2-C₃ alkyl carbonyl. In an exemplary embodiment, R¹ is acetyl.

In an exemplary embodiment, R² is an amino acid sequence which is complementary to a first specific binding member described herein. In an exemplary embodiment, R² is an amino acid sequence which is complementary to a first specific binding region described herein.

In an exemplary embodiment, the complementary binding member has an IC50 of between about 1 and about 10 uM for the first specific binding region. In an exemplary embodiment, the complementary binding member has an IC50 of between about 1 and about 10 uM for a first specific binding region described herein. In an exemplary embodiment, the complementary binding member has an IC50 of between about 1 and about 10 uM for a variable heavy domain described herein. In an exemplary embodiment, the complementary binding member has an IC50 of between about 1 and about 10 uM for a variable light domain described herein. In an exemplary embodiment, the complementary binding member has an IC50 of between about 1 and about 10 uM for a vlCDR1, or a vlCDR2, or a vlCDR3, or a vhCDR1, or a vhCDR2, or a vhCDR3 described herein. The IC50 of the complimentary bind region can be determined using a standard ELISA (enzyme-linked immunosorbent assay). Specifically, a target protein or control complementary binding member (i.e EGFR for cetuximab) is captured on a microtiter plate using reagent such as 0.2 M NaBicarbonate pH 9.6. A solution of target antibody or first specific binding member (i.e. cetuximab) is then added to the microtiter plate followed by washing steps. A secondary antibody (i.e goat anti-human IgG) that bind the constant region of cetuximab conjugated to a reporter enzyme (HRP) is then added. After washing HRP activity is detected using HRP detection reagents (colored or fluorescent) which are available from many comercial provided including Pierce or ThermoScientific. The IC50 of a complimentary binding region can be determined by additionally adding the complementary binding molecule together with the solution of target antibody at varied concentrations. Concentrations are usually tested over a 6 log range depending or expected IC50. Example concentration could be no complementary binding region, versus 0.001, 0.01, 0.1, 1.0, 10.0, 100.0 uM. Results are then plotted on a log scale to determine concentration that shows 50 percent inhibition of antibody (i.e cetuximab) binding to antigen (i.e. EGFR). A very high affinity complimentary binding member can be used as a positive control for 100 percent inhibition.

R² Complementary to Cetuximab

In an exemplary embodiment, R² is QGQSGQCISPRGCPDGPYVMY (SEQ ID NO:25). In an exemplary embodiment, R² is at least 90%, or at least 92%, or at least 94%, or at least 96%, or at least 98%, sequence identity to QGQSGQCISPRGCPDGPYVMY (SEQ ID NO:25).

R² Complementary to Trastuzumab

In an exemplary embodiment, R² is GSGSGSQLGPYELWELSHGSGS (SEQ ID NO:26). In an exemplary embodiment, R² is at least 90%, or at least 92%, or at least 94%, or at least 96%, or at least 98%, sequence identity to GSGSGSQLGPYELWELSHGSGS (SEQ ID NO:26).

In an exemplary embodiment, R² is QVSHWVSGLAEGSFG (SEQ ID NO:27). In an exemplary embodiment, R² is at least 90%, or at least 92%, or at least 94%, or at least 96%, or at least 98%, sequence identity to QVSHWVSGLAEGSFG (SEQ ID NO:27).

In an exemplary embodiment, R² is LSHTSGRVEGSVSLL (SEQ ID NO:28). In an exemplary embodiment, R² is at least 90%, or at least 92%, or at least 94%, or at least 96%, or at least 98%, sequence identity to LSHTSGRVEGSVSLL (SEQ ID NO:28).

R² Complementary to Adalimumab

In an exemplary embodiment, R² is HIHDDLLRYYGW (SEQ ID NO:29). In an exemplary embodiment, R² is at least 90%, or at least 92%, or at least 94%, or at least 96%, or at least 98%, sequence identity to HIHDDLLRYYGW (SEQ ID NO:29).

In an exemplary embodiment, R⁴ comprises a protease cleavage substrate. In an exemplary embodiment, R⁴ is a protease cleavage substrate. In an exemplary embodiment, R⁴ comprises a protease cleavage substrate described herein. In an exemplary embodiment, R⁴ is a protease cleavage substrate described herein.

In an exemplary embodiment, R⁴ comprises a matrix metalloproteinase cleavage substrate. In an exemplary embodiment, R⁴ is a matrix metalloproteinase cleavage substrate. In an exemplary embodiment, R⁴ comprises PLGLAG (SEQ ID NO:30) or PLGC(met)AG (SEQ ID NO:31). In an exemplary embodiment, R⁴ is PLGLAG (SEQ ID NO:30) or PLGC(met)AG (SEQ ID NO:31). In an exemplary embodiment, R⁴ comprises RS-(Cit)-G-(homoF)-YLY (SEQ ID NO:32), CRPAHLRDSG (SEQ ID NO:33), SLAYYTA (SEQ ID NO:34), NISDLTAG (SEQ ID NO:35), PPSSLRVT (SEQ ID NO:36), SGESLSNLTA (SEQ ID NO:37), or RIGFLR (SEQ ID NO:38). In an exemplary embodiment, R⁴ is RS-(Cit)-G-(homoF)-YLY (SEQ ID NO:32), CRPAHLRDSG (SEQ ID NO:33), SLAYYTA (SEQ ID NO:34), NISDLTAG (SEQ ID NO:35), PPSSLRVT (SEQ ID NO:36), SGESLSNLTA (SEQ ID NO:37), or RIGFLR (SEQ ID NO:38).

In an exemplary embodiment, R⁴ comprises a matrix metalloproteinase 2 cleavage substrate. In an exemplary embodiment, R⁴ is a matrix metalloproteinase 2 cleavage substrate. In an exemplary embodiment, R⁴ comprises TLSE-LH (SEQ ID NO:39) or TIAHLA (SEQ ID NO:40). In an exemplary embodiment, R⁴ is TLSE-LH (SEQ ID NO:39) or TIAHLA (SEQ ID NO:40).

In an exemplary embodiment, R⁴ comprises a matrix metalloproteinase 9 cleavage substrate. In an exemplary embodiment, R⁴ is a matrix metalloproteinase 9 cleavage substrate. In an exemplary embodiment, R⁴ comprises SNPYK-Y (SEQ ID NO:41), SNPKG-Y (SEQ ID NO:42), or SNPYG-Y (SEQ ID NO:43). In an exemplary embodiment, R⁴ is SNPYK-Y (SEQ ID NO:41), SNPKG-Y (SEQ ID NO:42), or SNPYG-Y (SEQ ID NO:43).

In an exemplary embodiment, R⁴ comprises a matrix metalloproteinase 14 cleavage substrate. In an exemplary embodiment, R⁴ is a matrix metalloproteinase 14 cleavage substrate. In an exemplary embodiment, R⁴ comprises RSHP(Hfe)TLY (SEQ ID NO:44) or RSHG(Hfe)FLY (SEQ ID NO:45). In an exemplary embodiment, R⁴ is RSHP(Hfe)TLY (SEQ ID NO:44) or RSHG(Hfe)FLY (SEQ ID NO:45).

In an exemplary embodiment, R⁴ comprises a cathepsin K cleavage substrate. In an exemplary embodiment, R⁴ is a cathepsin K cleavage substrate. In an exemplary embodiment, R⁴ comprises KLRFSKQ (SEQ ID NO:46). In an exemplary embodiment, R⁴ is KLRFSKQ (SEQ ID NO:46).

In an exemplary embodiment, R⁴ comprises a plasminogen cleavage substrate. In an exemplary embodiment, R⁴ is a plasminogen cleavage substrate.

In an exemplary embodiment, R⁴ comprises a plasmin cleavage substrate. In an exemplary embodiment, R⁴ is a plasmin cleavage substrate. In an exemplary embodiment, R⁴ comprises RLQLKL (SEQ ID NO:47). In an exemplary embodiment, R⁴ is RLQLKL (SEQ ID NO:47).

In an exemplary embodiment, R⁴ comprises a urokinase plasminogen activator cleavage substrate. In an exemplary embodiment, R⁴ is a urokinase plasminogen activator cleavage substrate. In an exemplary embodiment, R⁴ comprises a tissue plasminogen activator cleavage substrate. In an exemplary embodiment, R⁴ is a tissue plasminogen activator cleavage substrate.

In an exemplary embodiment, R⁴ comprises YGRAAA (SEQ ID NO:48) or YGPRNR (SEQ ID NO:49). In an exemplary embodiment, R⁴ is YGRAAA (SEQ ID NO:48) or YGPRNR (SEQ ID NO:49).

In an exemplary embodiment, R⁴ comprises a thrombin cleavage substrate. In an exemplary embodiment, R⁴ is a thrombin cleavage substrate. In an exemplary embodiment, R⁴ comprises DPRSFL (SEQ ID NO:50), PPRSFL (SEQ ID NO:51), TRPSFL (SEQ ID NO:52), or Norleucine-TPRSFL (SEQ ID NO:53). In an exemplary embodiment, R⁴ is DPRSFL (SEQ ID NO:50), PPRSFL (SEQ ID NO:51), TRPSFL (SEQ ID NO:52), or Norleucine-TPRSFL (SEQ ID NO:53).

In an exemplary embodiment, R⁴ comprises an elastase cleavage substrate. In an exemplary embodiment, R⁴ is an elastase cleavage substrate. In an exemplary embodiment, R⁴ comprises RLQLK(acetyl)L (SEQ ID NO:54) or RLQLA(acetyl)L (SEQ ID NO:55). In an exemplary embodiment, R⁴ is RLQLK(acetyl)L (SEQ ID NO:54) or RLQLA(acetyl)L (SEQ ID NO:55).

In an exemplary embodiment, R⁴ comprises a chymase cleavage substrate. In an exemplary embodiment, R⁴ is a chymase cleavage substrate. In an exemplary embodiment, R⁴ comprises GVAYSGA (SEQ ID NO:56). In an exemplary embodiment, R⁴ is GVAYSGA (SEQ ID NO:56).

In an exemplary embodiment, R⁴ comprises a peroxide cleavage substrate. In an exemplary embodiment, R⁴ is a peroxide cleavage substrate. In an exemplary embodiment, R⁴ comprises a hydrogen peroxide cleavage substrate. In an exemplary embodiment, R⁴ is a hydrogen peroxide cleavage substrate. In an exemplary embodiment, R⁴ comprises ACPP1 and/or ACPP2. In an exemplary embodiment, R⁴ is ACPP1 and/or ACPP2. The representative structure for ACPP1 is:

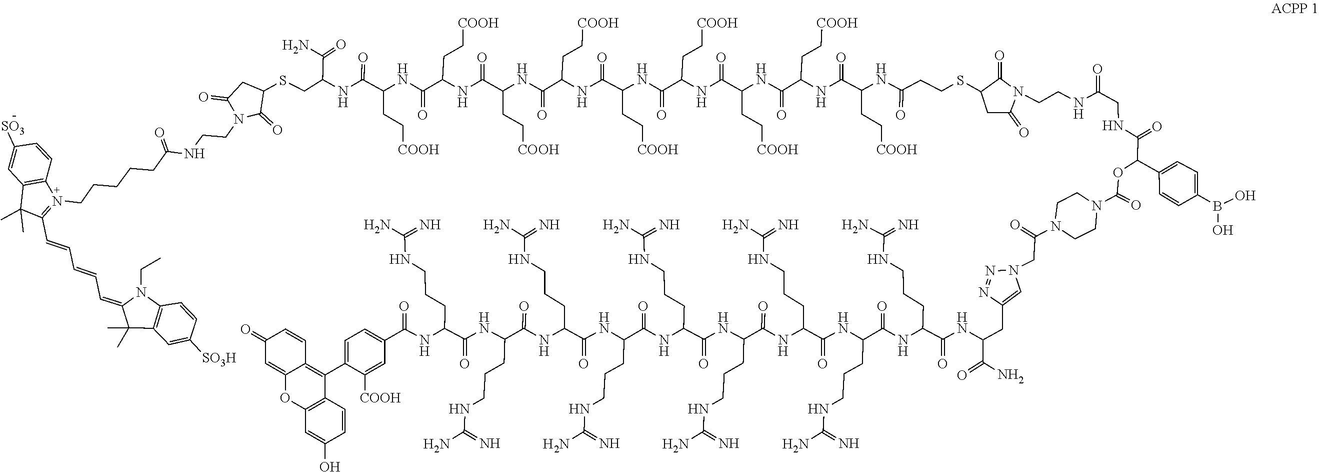

The representative structure for ACPP2 is:

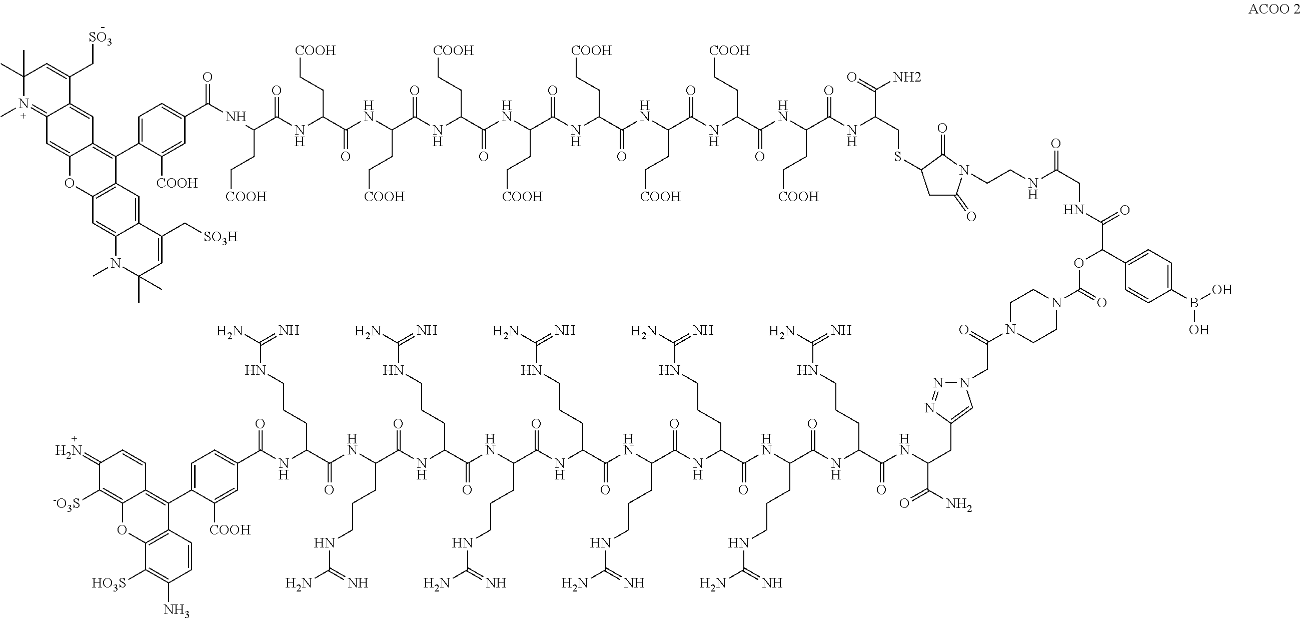

Other related structures cleavable by hydrogen peroxide are also contemplated by the present invention.

In an exemplary embodiment, m is 0 and n is 0. In an exemplary embodiment, m is 1 and n is 0. In an exemplary embodiment, m is 0 and n is 1. In an exemplary embodiment, m is 1 and n is 1.

In an exemplary embodiment, the R³ and the R⁵ each independently comprise a member selected from polyalkylene oxide. In an exemplary embodiment, the R³ and the R⁵ each independently comprise a member selected from polypropylene oxide or polyethylene oxide. In an exemplary embodiment, the R³ and the R⁵ each independently comprise a member selected from linear polyalkylene oxide or branched polyalkylene oxide. In an exemplary embodiment, the R³ and the R⁵ each independently comprise a member selected from the group consisting of linear polypropylene oxide, branched polypropylene oxide, linear polyethylene oxide, and branched polyethylene oxide.

In an exemplary embodiment, m is 1 and R³ is PEG with between 2 and 50 subunits. In an exemplary embodiment, m is 1 and R³ is PEG with between 2 and 10 subunits. In an exemplary embodiment, m is 1 and R³ is PEG with between 4 and 8 subunits. In an exemplary embodiment, m is 1 and R³ is PEG with between 8 and 20 subunits. In an exemplary embodiment, m is 1 and R³ is PEG with between 10 and 20 subunits. In an exemplary embodiment, m is 1 and R³ is PEG with between 15 and 25 subunits. In an exemplary embodiment, m is 1 and R³ is PEG with between 20 and 30 subunits. In an exemplary embodiment, m is 1 and R³ is PEG with between 25 and 35 subunits. In an exemplary embodiment, m is 1 and R³ is PEG with between 30 and 40 subunits. In an exemplary embodiment, m is 1 and R³ is PEG with between 35 and 45 subunits. In an exemplary embodiment, m is 1 and R³ is PEG with between 40 and 50 subunits. In an exemplary embodiment, m is 1 and R³ is 1 Angstrom to 200 Angstroms in length. In an exemplary embodiment, m is 1 and R³ is 1 Angstrom to 150 Angstroms in length. In an exemplary embodiment, m is 1 and R³ is 10 Angstrom to 120 Angstroms in length. In an exemplary embodiment, m is 1 and R³ is 50 Angstrom to 150 Angstroms in length. In an exemplary embodiment, m is 1 and R³ is 1 Angstrom to 200 Angstroms in length.

In an exemplary embodiment, n is 1 and R⁵ is PEG with between 2 and 50 subunits. In an exemplary embodiment, n is 1 and R⁵ is PEG with between 2 and 10 subunits. In an exemplary embodiment, n is 1 and R⁵ is PEG with between 4 and 8 subunits. In an exemplary embodiment, n is 1 and R⁵ is PEG with between 8 and 20 subunits. In an exemplary embodiment, n is 1 and R⁵ is PEG with between 10 and 20 subunits. In an exemplary embodiment, n is 1 and R⁵ is PEG with between 15 and 25 subunits. In an exemplary embodiment, n is 1 and R⁵ is PEG with between 20 and 30 subunits. In an exemplary embodiment, n is 1 and R⁵ is PEG with between 25 and 35 subunits. In an exemplary embodiment, n is 1 and R⁵ is PEG with between 30 and 40 subunits. In an exemplary embodiment, n is 1 and R⁵ is PEG with between 35 and 45 subunits. In an exemplary embodiment, n is 1 and R⁵ is PEG with between 40 and 50 subunits. In an exemplary embodiment, n is 1 and R⁵ is 1 Angstrom to 200 Angstroms in length. In an exemplary embodiment, n is 1 and R⁵ is 1 Angstrom to 150 Angstroms in length. In an exemplary embodiment, n is 1 and R⁵ is 10 Angstrom to 120 Angstroms in length. In an exemplary embodiment, n is 1 and R⁵ is 50 Angstrom to 150 Angstroms in length. In an exemplary embodiment, n is 1 and R⁵ is 1 Angstrom to 200 Angstroms in length.

In an exemplary embodiment, (R³)ₘ—R⁴—(R⁵)ₙ—R⁶ is from 1 Angstrom to 200 Angstroms in length. In an exemplary embodiment, (R³)ₘ—R⁴—(R⁵)ₙ—R⁶ is from 1 Angstrom to 150 Angstroms in length. In an exemplary embodiment, (R³)ₘ—R⁴—(R⁵)ₙ—R⁶ is from 10 Angstrom to 120 Angstroms in length. In an exemplary embodiment, (R³)ₘ—R⁴—(R⁵)ₙ—R⁶ is from 50 Angstrom to 150 Angstroms in length. In an exemplary embodiment, (R³)ₘ—R⁴—(R⁵)ₙ—R⁶ is from 1 Angstrom to 200 Angstroms in length. In an exemplary embodiment, (R³)ₘ—R⁴—(R⁵)ₙ—R⁶ is from 1 Angstrom to 200 Angstroms in length. In an exemplary embodiment, the R⁵ is 1 Angstrom to 150 Angstroms in length. In an exemplary embodiment, the R⁵ is 10 Angstrom to 120 Angstroms in length. In an exemplary embodiment, the R⁵ is 50 Angstrom to 150 Angstroms in length. In an exemplary embodiment, the R⁵ is 1 Angstrom to 200 Angstroms in length.

In an exemplary embodiment, the complementary binding member/linker has a structure according to: R¹—R²—(R³)ₘ—R⁴—(R⁵)ₙ—R⁶, wherein m is 0, n is 1 and R⁵ is PEG with between 5 and 50 subunits, or between 5 and 15 subunits, or between 10 and 20 subunits, or between 15 and 25 subunits, or between 20 and 30 subunits, or between 25 and 35 subunits, or between 30 and 40 subunits, or between 35 and 45 subunits, or between 40 and 50 subunits, or between 15 and 40 subunits, or between 10 and 30 subunits, or between 20 and 45 subunits. In an exemplary embodiment, the complementary binding member/linker has a structure according to: R¹—R²—(R³)ₘ—R⁴—(R⁵)ₙ—R⁶, wherein m is 1, n is 0 and R³ is PEG with between 5 and 50 subunits, or between 5 and 15 subunits, or between 10 and 20 subunits, or between 15 and 25 subunits, or between 20 and 30 subunits, or between 25 and 35 subunits, or between 30 and 40 subunits, or between 35 and 45 subunits, or between 40 and 50 subunits, or between 15 and 40 subunits, or between 10 and 30 subunits, or between 20 and 45 subunits.

In an exemplary embodiment, the complementary binding member/linker has a structure according to: R¹—R²—(R³)ₘ—R⁴—(R⁵)ₙ—R⁶, wherein m is 1, n is 1, R¹, R², R⁴, R⁶ are as described herein, and R³ is PEG with between 1 and 5 subunits and R⁵ is PEG with between 1 and 5 subunits, or R³ is PEG with between 6 and 10 subunits and R⁵ is PEG with between 1 and 5 subunits, or R³ is PEG with between 1 and 5 subunits and R⁵ is PEG with between 6 and 10 subunits, or R³ is PEG with between 1 and 20 subunits and R⁵ is PEG with between 1 and 20 subunits, or R³ is PEG with between 1 and 40 subunits and R⁵ is PEG with between 1 and 40 subunits, or R³ is PEG with between 5 and 20 subunits and R⁵ is PEG with between 5 and 20 subunits, or R³ is PEG with between 10 and 20 subunits and R⁵ is PEG with between 1 and 10 subunits, or R³ is PEG with between 1 and 10 subunits and R⁵ is PEG with between 10 and 20 subunits, or R³ is PEG with between 5 and 30 subunits and R⁵ is PEG with between 5 and 30 subunits, or R³ is PEG with between 5 and 15 subunits and R⁵ is PEG with between 15 and 30 subunits, or R³ is PEG with between 15 and 30 subunits and R⁵ is PEG with between 5 and 15 subunits

In an exemplary embodiment, the first linker site is a lysine, and prior to conjugation with the first linker site, R⁶ is a reactive functional group capable of forming a covalent bond with the nitrogen on the side chain of lysine. In an exemplary embodiment, the first linker site is a lysine, and prior to conjugation with the first linker site, R⁶ is an NHS ester. In an exemplary embodiment, the first linker site is a lysine, and prior to conjugation with the first linker site, R⁶ is an imidoester. In an exemplary embodiment, the first linker site is a lysine, and prior to conjugation with the first linker site, R⁶ is isothiocyanate, isocyanate, acyl azide, sulfonyl chloride, aldehyde, glyoxal, epoxide, oxirane, carbonate, aryl halide, carbodiimide, anhydride, or fluorophenyl ester.

In one embodiment, the first linker site is a lysine, and prior to conjugation with the first linker site, R⁶ is a reactive functional group which is an N-hydroxysuccinimide (NHS) ester, sulfur-NHS ester, imidoester, isocyanate, isothiocyanate, acylhalide, arylazide, p-nitrophenyl ester, aldehyde, sulfonyl chloride, thiazolide or carboxyl group.

NHS esters and sulfur-NHS esters react preferentially with a primary (including aromatic) amino groups of a reaction partner. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide.

Imidoesters are the most specific acylating reagents for reaction with amine groups of a molecule such as a protein. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with the primary amines of the conjugate components to form stable bonds. Their reactions with sulfhydryl, imidazole, and tyrosyl groups give relatively unstable products.

Acylazides are also used as amino-specific reagents in which nucleophilic amines of the reaction partner attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of the conjugate components, but also with its sulfhydryl and imidazole groups.

p-Nitrophenyl esters of carboxylic acids are also useful amino-reactive groups. Although the reagent specificity is not very high, α- and ε-amino groups appear to react most rapidly.

Aldehydes react with primary amines of the conjugate components (e.g., ε-amino group of lysine residues). Although unstable, Schiff bases are formed upon reaction of the protein amino groups with the aldehyde. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product. Alternatively, a stable bond may be formed by reductive amination.

Aromatic sulfonyl chlorides react with a variety of sites of the conjugate components, but reaction with the amino groups is the most important, resulting in a stable sulfonamide linkage.

Free carboxyl groups react with carbodiimides, soluble in both water and organic solvents, forming pseudoureas that can then couple to available amines yielding an amide linkage. Yamada et al., Biochemistry, 1981, 20: 4836-4842, e.g., teach how to modify a protein with carbodiimides.

In an exemplary embodiment, the first linker site is a cysteine, and prior to conjugation with the first linker site, R⁶ is a reactive functional group capable of forming a covalent bond with the sulfur on the side chain of cysteine. In an exemplary embodiment, the first linker site is a cysteine, and prior to conjugation with the first linker site, R⁶ is a reactive functional group which is maleimide. In an exemplary embodiment, the first linker site is a cysteine, and prior to conjugation with the first linker site, R⁶ is a reactive functional group which is haloacetyl. In an exemplary embodiment, the first linker site is a cysteine, and prior to conjugation with the first linker site, R⁶ is a reactive functional group which is aziridine, acryloyl, arylating agent, vinylsulfone, pyridyl disulfide, TNB-thiol, 5,5′-dithiobis-(2-nitrobenzoic acid), or disulfide reducing agent.

In one embodiment, the first linker site is a cysteine, and prior to conjugation with the first linker site, R⁶ is a reactive functional group which is a maleimide, alkyl halide, acyl halide (including bromoacetamide or chloroacetamide), pyridyl disulfide, and thiophthalimide.

Maleimides react preferentially with the sulfhydryl group of the conjugate components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryl groups via disulfide exchange to give mixed disulfides. As a result, pyridyl disulfides are relatively specific sulfhydryl-reactive groups.

Thiophthalimides react with free sulfhydryl groups to also form disulfides.

In an exemplary embodiment, the first linker site is lysine and the complementary binding member/linker has a structure according to the following formula:

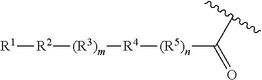

wherein [symbol] is the point of attachment to the nitrogen on the side chain of the lysine of the first linker site.

In an exemplary embodiment, the first linker site is lysine and the complementary binding member/linker has a structure according to the following formula:

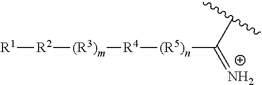

wherein [symbol] is the point of attachment to the nitrogen on the side chain of the lysine of the first linker site.

In an exemplary embodiment, the first linker site is cysteine and the complementary binding member/linker has a structure according to the following formula:

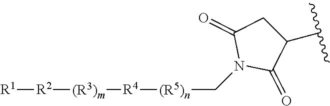

wherein [symbol] is the point of attachment to the sulfur on the side chain of the cysteine of the first linker site.

In an exemplary embodiment, the first linker site is cysteine and the complementary binding member/linker has a structure according to the following formula:

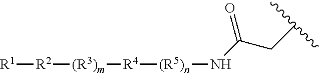

wherein [symbol] is the point of attachment to the sulfur on the side chain of the cysteine of the first linker site.

Methods of attachment of PEG to amino acids are known to one of skill in the art and described in references such as Bioconjugate Techniques, 3rd Edition by Greg T. Hermanson (Academic Press, 2013).

In an exemplary embodiment, the target binding domain comprises EGFR. In an exemplary embodiment, the target binding domain comprises the extracellular domain of EGFR. In an exemplary embodiment, the target binding domain comprises HER-2. In an exemplary embodiment, the target binding domain comprises TNFα.

In an exemplary embodiment, the target binding domain comprises human EGFR. In an exemplary embodiment, the target binding domain comprises the extracellular domain of human EGFR. In an exemplary embodiment, the target binding domain comprises human HER-2. In an exemplary embodiment, the target binding domain comprises human TNFα.

In an exemplary embodiment, the linker of the activatable specific binding member complex allows specific binding and reversible binding of said first specific binding region with said first complementary binding member. In an exemplary embodiment, the linker is cleaved at said cleavable substrate, said first specific binding member and said first complementary binding member become capable of dissociating from each other. In an exemplary embodiment, the linker is cleaved at said cleavable substrate, said first specific binding region and said first complementary binding member dissociate from each other. In an exemplary embodiment, the linker is cleaved at said cleavable substrate, said first specific binding region and said first complementary binding member dissociate from each other, thus forming an activated specific binding member; and wherein said activated specific binding member functions such that said first specific binding region can bind with at least one moiety other than said first complementary binding member. In an exemplary embodiment, the at least one moiety other than said first complementary binding member is a first target binding domain. In an exemplary embodiment, the first specific binding member comprises between about 25% and about 99% of a CDR of an antibody selected from the group consisting of adalimumab, bezlotoxumab, avelumab, dupilumab, durvalumab, brodalumab, reslizumab, olaratumab, daratumumab, elotuzumab, necitumumab, infliximab, obiltoxaximab, atezolizumab, secukinumab, mepolizumab, nivolumab, alirocumab, idarucizumab, evolocumab, dinutuximab, bevacizumab, pembrolizumab, ramucirumab, vedolizumab, siltuximab, alemtuzumab, trastuzumab emtansine, pertuzumab, infliximab, obinutuzumab, brentuximab, raxibacumab, belimumab, ipilimumab, denosumab, ofatumumab, besilesomab, tocilizumab, canakinumab, golimumab, ustekinumab, certolizumab pegol, catumaxomab, eculizumab, ranibizumab, panitumumab, natalizumab, bevacizumab, omalizumab, cetuximab, efalizumab, ibritumomab tiuxetan, fanolesomab, adalimumab, tositumomab, iodine 131 tositumomab, alemtuzumab, trastuzumab, gemtuzumab ozogamicin, infliximab, palivizumab, necitumumab, basiliximab, rituximab, votumumab, sulesomab, arcitumomab, imiciromab, capromab, nofetumomab, and abciximab. In an exemplary embodiment, the first specific binding member comprises between about 25% and about 99% of a CDR of an antibody selected from the group consisting of cetuximab, trastuzumab, or adalimumab. In an exemplary embodiment, the first linker site is a lysine or a cysteine. In an exemplary embodiment, said R⁴ comprises an uPA cleavage substrate, an MMP cleavage substrate, or a thrombin cleavage substrate. In an exemplary embodiment, when m is 1, R³ comprises a member selected from the group consisting of PEG, a protein nucleic acid (PNA), a D amino acid, an L amino acid, a lipophilic residue, an SPDB disulfide, MCC (maleimidomethyl cyclohexane-1-carboxylate), sulfo-SPDB which adds a charged polar group, hydrazine, and combinations thereof. In an exemplary embodiment, when m is 1, R³ is PEG. In an exemplary embodiment, when n is 1, R⁵ comprises a member selected from the group consisting of PEG, a protein nucleic acid (PNA), a D amino acid, an L amino acid, a lipophilic residue, an SPDB disulfide, MCC (maleimidomethyl cyclohexane-1-carboxylate), sulfo-SPDB which adds a charged polar group, hydrazine, and combinations thereof. In an exemplary embodiment, when n is 1, R⁵ is PEG. In an exemplary embodiment, wherein said first target binding domain comprises a member selected from the group consisting of EGFR, HER-2, VEGF, CD20, CTLA-1 PDL-1, C. difficile toxin B, TNFα, PD-L1, IL-4Rα, CD20, IL-17RA, IL-5, PDGFR-α, D38, SLAMF7, EGFR,PA component of B. anthracis toxin, interleukin-17A, IL-5, PD-1, PCSK9, dabigatran etexilate, LDL-C/PCSK9, GD2, CD19, VEGF, Integrin-α4β7, cCLB8, CD52, HER2, CD30, Bacillus anthracis protective antigen, BLyS, CTLA-4, RANKL, NCA-95, IL-6 receptor, IL-1B, IL-12/IL-23, EpCAM and CD3, Complement C5, VLA-4, EpCAM, IgE, CD11a, CD15, CD33, F-protein of RS virus, CD25 (a chain of IL2 receptor), Cytokeratintumor-associated antigen, Human cardiac myosin, NCA90, Human CEA (carcinoembryonic antigen), Tumor surface antigen PSMA, Carcinoma-associated antigen GPIIb/IIIa, integrins, an antibody drug target, any cell determinant, or a combination thereof. In an exemplary embodiment, said first target binding domain comprises a member selected from the group consisting of EGFR, HER-2, and TNFα. In an exemplary embodiment, the first specific binding region comprises between about 25% and about 99% of a CDR of cetuximab, said first target binding domain is EGFR, said first linker site is lysine or cysteine.

In an exemplary embodiment, the invention provides a composition comprising activatable specific binding member complexes described herein. In an exemplary embodiment, the invention provides a composition comprising activatable specific binding member complexes, comprising: a) a first specific binding member, comprising: 1) a first specific binding region, with binding affinity for a first target binding domain; 2) a first linker site which is a lysine; and b) a complementary binding member/linker according to the following formula: R¹—R²—(R³)ₘ—R⁴—(R⁵)ₙ—R⁶, wherein R¹ is substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl; R² is a first complementary binding member; R³ is a first sublinker; m is either 0 or 1; R⁴ is a cleavable substrate; R⁵ is a second sublinker; n is either 0 or 1; R⁶ is the attachment point to the first linker site of the first specific binding member.

In an exemplary embodiment, the invention provides a composition comprising activatable specific binding member complexes, comprising: a) a first specific binding member, comprising: 1) a first specific binding region, with binding affinity for a first target binding domain; 2) a first linker site which is a cysteine; and b) a complementary binding member/linker according to the following formula: R¹—R²—(R³)ₘ—R⁴—(R⁵)ₙ—R⁶, wherein R¹ is substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl; R² is a first complementary binding member; R³ is a first sublinker; m is either 0 or 1; R⁴ is a cleavable substrate; R⁵ is a second sublinker; n is either 0 or 1; R⁶ is the attachment point to the first linker site of the first specific binding member.

In an exemplary embodiment, the invention provides a composition comprising activatable specific binding member complexes, prepared by a process comprising: a) a first specific binding member, comprising: 1) a first specific binding region, with binding affinity for a first target binding domain; 2) a first linker site which is a cysteine; and b) a complementary binding member/linker according to the following formula: R¹—R²—(R³)ₘ—R⁴—(R⁵)ₙ—R⁶, wherein R¹ is substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl; R² is a first complementary binding member; R³ is a first sublinker; m is either 0 or 1; R⁴ is a cleavable substrate; R⁵ is a second sublinker; n is either 0 or 1; R⁶ is the attachment point to the first linker site of the first specific binding member.

### Cetuximab Embodiments:

In an exemplary embodiment, the activatable specific binding member complex, comprises a first specific binding member which is a cetuximab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is a peptide sequence with an IC50 for a CDR of cetuximab described herein of between about 1 and about 10 uM.

In an exemplary embodiment, the activatable specific binding member complex, comprises a first specific binding member which is a cetuximab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is a peptide sequence with an IC50 for a CDR of cetuximab of between about 1 and about 10 uM.

In an exemplary embodiment, the activatable specific binding member complex, comprises a first specific binding member which is a cetuximab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to QGQSGQCISPRGCPDGPYVMY.

In an exemplary embodiment, the activatable specific binding member complex, comprises a first specific binding member which is a cetuximab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to QGQSGQCISPRGCPDGPYVMY.

In an exemplary embodiment, the activatable specific binding member complex, comprises a first specific binding member which is a cetuximab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁵, and R⁶ are as described herein; and R² is a peptide sequence with an IC50 for a CDR of cetuximab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate.

In an exemplary embodiment, the activatable specific binding member complex, comprises a first specific binding member which is a cetuximab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁵, and R⁶ are as described herein; and R² is a peptide sequence with an IC50 for a CDR of cetuximab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate.

In an exemplary embodiment, the first linker site is lysine and the complementary binding member/linker has a structure according to the following formula:

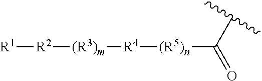

wherein [symbol] is the point of attachment to the nitrogen on the side chain of the lysine of the first linker site.

In an exemplary embodiment, the first linker site is lysine and the complementary binding member/linker has a structure according to the following formula:

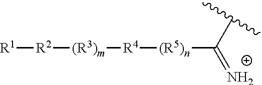

wherein [symbol] is the point of attachment to the nitrogen on the side chain of the lysine of the first linker site.

In an exemplary embodiment, the activatable specific binding member complex, comprises a first specific binding member which is a cetuximab embodiment described herein, and the first linker site is lysine and the complementary binding member/linker has a structure according to the following formula:

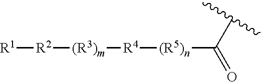

wherein m, n, R¹, R³, R⁵, and R⁶ are as described herein; and R² is a peptide sequence with an IC50 for a CDR of cetuximab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the nitrogen on the side chain of the lysine of the first linker site.

In an exemplary embodiment, the activatable specific binding member complex, comprises a first specific binding member which is a cetuximab embodiment described herein, and the first linker site is lysine and the complementary binding member/linker has a structure according to the following formula:

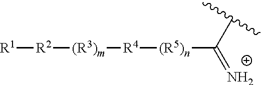

wherein m, n, R¹, R³, R⁵, and R⁶ are as described herein; and R² is a peptide sequence with an IC50 for a CDR of cetuximab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the nitrogen on the side chain of the lysine of the first linker site.

In an exemplary embodiment, the activatable specific binding member complex, comprises a first specific binding member which is a cetuximab embodiment described herein, and the first linker site is cysteine, the first linker site is cysteine and the complementary binding member/linker has a structure according to the following formula:

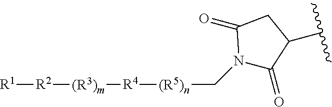

wherein m, n, R¹, R³, R⁵, and R⁶ are as described herein; and R² is a peptide sequence with an IC50 for a CDR of cetuximab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the sulfur on the side chain of the cysteine of the first linker site.

In an exemplary embodiment, the activatable specific binding member complex, comprises a first specific binding member which is a cetuximab embodiment described herein, and the first linker site is cysteine, the first linker site is cysteine and the complementary binding member/linker has a structure according to the following formula:

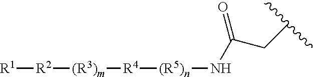

wherein m, n, R¹, R³, R⁵, and R⁶ are as described herein; and R² is a peptide sequence with an IC50 for a CDR of cetuximab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the sulfur on the side chain of the cysteine of the first linker site.

### Trastuzumab Embodiments:

In an exemplary embodiment, the activatable specific binding member complex, comprises a first specific binding member which is a trastuzumab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to GSGSGSQLGPYELWELSHGSGS.

In an exemplary embodiment, the activatable specific binding member complex, comprises a first specific binding member which is a trastuzumab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to GSGSGSQLGPYELWELSHGSGS.

In an exemplary embodiment, the activatable specific binding member complex, comprises a first specific binding member which is a trastuzumab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to QVSHWVSGLAEGSFG.

In an exemplary embodiment, the activatable specific binding member complex, comprises a first specific binding member which is a trastuzumab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to QVSHWVSGLAEGSFG.

In an exemplary embodiment, the activatable specific binding member complex, comprises a first specific binding member which is a trastuzumab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to LSHTSGRVEGSVSLL.

In an exemplary embodiment, the activatable specific binding member complex, comprises a first specific binding member which is a trastuzumab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to LSHTSGRVEGSVSLL.

In an exemplary embodiment, the activatable specific binding member complex, comprises a first specific binding member which is a trastuzumab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁵, and R⁶ are as described herein; and R² is a peptide sequence with an IC50 for a CDR of trastuzumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate.

In an exemplary embodiment, the activatable specific binding member complex, comprises a first specific binding member which is a trastuzumab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁵, and R⁶ are as described herein; and R² is a peptide sequence with an IC50 for a CDR of trastuzumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate.

In an exemplary embodiment, the activatable specific binding member complex, comprises a first specific binding member which is a trastuzumab embodiment described herein, and the first linker site is lysine and the complementary binding member/linker has a structure according to the following formula:

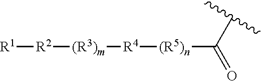

wherein m, n, R¹, R³, R⁵, and R⁶ are as described herein; and R² is a peptide sequence with an IC50 for a CDR of trastuzumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the nitrogen on the side chain of the lysine of the first linker site.

In an exemplary embodiment, the activatable specific binding member complex, comprises a first specific binding member which is a trastuzumab embodiment described herein, and the first linker site is lysine and the complementary binding member/linker has a structure according to the following formula:

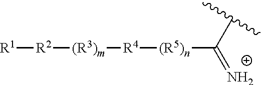

wherein m, n, R¹, R³, R⁵, and R⁶ are as described herein; and R is a peptide sequence with an IC50 for a CDR of trastuzumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the nitrogen on the side chain of the lysine of the first linker site.

In an exemplary embodiment, the activatable specific binding member complex, comprises a first specific binding member which is a trastuzumab embodiment described herein, and the first linker site is cysteine, the first linker site is cysteine and the complementary binding member/linker has a structure according to the following formula:

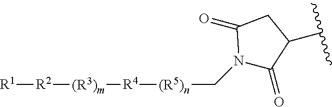

wherein m, n, R¹, R³, R⁵, and R⁶ are as described herein; and R² is a peptide sequence with an IC50 for a CDR of trastuzumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the sulfur on the side chain of the cysteine of the first linker site.

In an exemplary embodiment, the activatable specific binding member complex, comprises a first specific binding member which is a trastuzumab embodiment described herein, and the first linker site is cysteine, the first linker site is cysteine and the complementary binding member/linker has a structure according to the following formula:

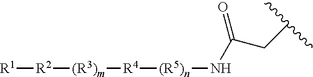

wherein m, n, R¹, R³, R⁵, and R⁶ are as described herein; and R² is a peptide sequence with an IC50 for a CDR of trastuzumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the sulfur on the side chain of the cysteine of the first linker site.

### Adalimumab Embodiments:

In an exemplary embodiment, the activatable specific binding member complex, comprises a first specific binding member which is an adalimumab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is a peptide sequence with an IC50 for a CDR of adalimumab described herein of between about 1 and about 10 uM.

In an exemplary embodiment, the activatable specific binding member complex, comprises a first specific binding member which is an adalimumab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is a peptide sequence with an IC50 for a CDR of adalimumab of between about 1 and about 10 uM.

In an exemplary embodiment, the activatable specific binding member complex, comprises a first specific binding member which is an adalimumab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to HIHDDLLRYYGW.

In an exemplary embodiment, the activatable specific binding member complex, comprises a first specific binding member which is an adalimumab embodiment described herein, and the first linker site is cysteine, and the complementary binding member/linker has the following formula:

R¹—R²—(R³)ₘ—R⁴—(R⁵)ₙ—R⁶

wherein m, n, R¹, R³, R⁴, R⁵, and R⁶ are as described herein; and R² is at least 90%, or at least 92%, or at least 94%, or at least 96%, or at least 98%, sequence identity to HIHDDLLRYYGW.

In an exemplary embodiment, the activatable specific binding member complex, comprises a first specific binding member which is an adalimumab embodiment described herein, and the first linker site is lysine, and the complementary binding member/linker has the following formula:

R¹—R²—(R³)ₘ—R⁴—(R⁵)ₙ—R⁶

wherein m, n, R¹, R³, R⁵, and R⁶ are as described herein; and R² is a peptide sequence with an IC50 for a CDR of adalimumab described herein of between about 1 and about 10 uM and R⁴ comprises a cleavage substrate described herein which a cathepsin K cleavage substrate, thrombin cleavage substrate, or chymase cleavage substrate.

In an exemplary embodiment, the activatable specific binding member complex, comprises a first specific binding member which is adalimumab, and the first linker site is cysteine, and the complementary binding member/linker has the following formula:

R¹—R²—(R³)ₘ—R⁴—(R⁵)ₙ—R⁶

wherein m, n, R¹, R³, R⁵, and R⁶ are as described herein; and R² is a peptide sequence with an IC50 for a CDR of adalimumab described herein of between about 1 and about 10 uM and R⁴ comprises a cleavage substrate described herein which a cathepsin K cleavage substrate, thrombin cleavage substrate, or chymase cleavage substrate.

In an exemplary embodiment, the activatable specific binding member complex, comprises a first specific binding member which is an adalimumab embodiment described herein, and the first linker site is lysine and the complementary binding member/linker has a structure according to the following formula:

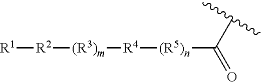

wherein m, n, R¹, R³, R⁵, and R⁶ are as described herein; and R² is a peptide sequence with an IC50 for a CDR of adalimumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the nitrogen on the side chain of the lysine of the first linker site.

In an exemplary embodiment, the activatable specific binding member complex, comprises a first specific binding member which is an adalimumab embodiment described herein, and the first linker site is lysine and the complementary binding member/linker has a structure according to the following formula:

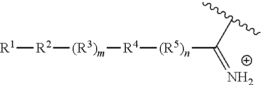

wherein m, n, R¹, R³, R⁵, and R⁶ are as described herein; and R² is a peptide sequence with an IC50 for a CDR of adalimumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the nitrogen on the side chain of the lysine of the first linker site.

In an exemplary embodiment, the activatable specific binding member complex, comprises a first specific binding member which is a adalimumab embodiment described herein, and the first linker site is cysteine, the first linker site is cysteine and the complementary binding member/linker has a structure according to the following formula:

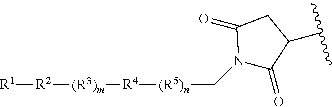

wherein m, n, R¹, R³, R⁵, and R⁶ are as described herein; and R² is a peptide sequence with an IC50 for a CDR of adalimumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the sulfur on the side chain of the cysteine of the first linker site.

In an exemplary embodiment, the activatable specific binding member complex, comprises a first specific binding member which is a adalimumab embodiment described herein, and the first linker site is cysteine, the first linker site is cysteine and the complementary binding member/linker has a structure according to the following formula:

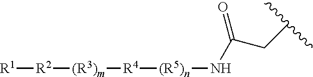

wherein m, n, R¹, R³, R⁵, and R⁶ are as described herein; and R² is a peptide sequence with an IC50 for a CDR of adalimumab described herein of between about 1 and about 10 uM and R⁴ comprises a cleavage substrate described herein which a matrix metalloproteinase 2 cleavage substrate, matrix metalloproteinase 9 cleavage substrate, matrix metalloproteinase 14 cleavage substrate, plasminogen cleavage substrate, plasmin cleavage substrate, urokinase plasminogen activator cleavage substrate, tissue plasminogen activator cleavage substrate, elastase cleavage substrate, or peroxide cleavage substrate, wherein [symbol] is the point of attachment to the sulfur on the side chain of the cysteine of the first linker site.

In an exemplary embodiment, the activatable specific binding member complex is produced by a process described herein. In an exemplary embodiment, the activatable specific binding member complex is produced by a process comprising contacting a first specific binding member described herein with a complementary binding member/linker according to the following formula: R¹—R²—(R³)ₘ—R⁴—(R⁵)ₙ—R⁶, wherein R⁶ is a reactive functional group described herein and the first linker site is compatible to react with the reactive functional group and form a covalent bond.

In an exemplary embodiment, the activatable specific binding member complex is produced by a process comprising contacting a first specific binding member described herein with a complementary binding member/linker according to the following formula: R¹—R²—(R³)ₘ—R⁴—(R⁵)ₙ—R⁶, wherein the first linker site is a lysine, and R⁶ comprises an isothiocyanate, isocyanate, acyl azide, sulfonyl chloride, aldehyde, glyoxal, epoxide, oxirane, carbonate, aryl halide, carbodiimide, anhydride, or fluorophenyl ester.

In an exemplary embodiment, the activatable specific binding member complex is produced by a process comprising contacting a first specific binding member described herein with a complementary binding member/linker according to the following formula: R¹—R²—(R³)ₘ—R⁴—(R⁵)ₙ—R⁶, wherein the first linker site is a lysine, and R⁶ comprises an NHS ester.

In an exemplary embodiment, the activatable specific binding member complex is produced by a process comprising contacting a first specific binding member described herein with a complementary binding member/linker according to the following formula: R¹—R²—(R³)ₘ—R⁴—(R⁵)ₙ—R⁶, wherein the first linker site is a cysteine, and R⁶ comprises a maleimide, alkyl halide, acyl halide (including bromoacetamide or chloroacetamide), pyridyl disulfide, or thiophthalimide.

In an exemplary embodiment, the activatable specific binding member complex is produced by a process comprising contacting a first specific binding member described herein with a complementary binding member/linker according to the following formula: R¹—R²—(R³)ₘ—R⁴—(R⁵)ₙ—R⁶, wherein the first linker site is a cysteine, and R⁶ comprises a maleimide.

In an exemplary embodiment, the activatable specific binding member complex is produced by a process comprising contacting a first specific binding member described herein with a complementary binding member/linker according to the following formula: R¹—R²—(R³)ₘ—R⁴—(R⁵)ₙ—R⁶, wherein the first linker site is a lysine, and R⁶ comprises an NHS ester.

The construction of expression vectors and the expression of genes in transfected cells involves the use of molecular cloning techniques that are well known in the art. See, for example, Sambrook et al., Molecular Cloning—A Laboratory Manual, Cold Spring Harbor Laboratory, Cold Spring Harbor, N.Y., (1989) and Current Protocols in Molecular Biology, F. M. Ausubel et al., eds., (Current Protocols, a joint venture between Greene Publishing Associates, Inc. and John Wiley & Sons, Inc., most recent Supplement). Nucleic acids used to transfect cells with sequences coding for expression of the polypeptide of interest generally will be in the form of an expression vector including expression control sequences operatively linked to a nucleotide sequence coding for expression of the polypeptide. As used herein, “operatively linked” refers to a juxtaposition wherein the components so described are in a relationship permitting them to function in their intended manner. A control sequence operatively linked to a coding sequence is ligated such that expression of the coding sequence is achieved under conditions compatible with the control sequences. “Control sequence” refers to polynucleotide sequences which are necessary to affect the expression of coding and non-coding sequences to which they are ligated. Control sequences generally include promoter, ribosomal binding site, and transcription termination sequence. The term “control sequences” is intended to include, at a minimum, components whose presence can influence expression, and can also include additional components whose presence is advantageous, for example, leader sequences and fusion partner sequences. As used herein, the term “nucleotide sequence coding for expression of a polypeptide refers to a sequence that, upon transcription and translation of mRNA, produces the polypeptide. This can include sequences containing, e.g., introns. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons.

Any suitable method is used to construct expression vectors containing the fluorescent indicator coding sequence and appropriate transcriptional/translational control signals. Any methods which are well known to those skilled in the art can be used to construct expression vectors containing the fluorescent indicator coding sequence and appropriate transcriptional/translational control signals. These methods include in vitro recombinant DNA techniques, synthetic techniques and in vivo recombination/genetic recombination. (See, for example, the techniques described in Maniatis, et al., Molecular Cloning A Laboratory Manual, Cold Spring Harbor Laboratory, N.Y., 1989). Transformation of a host cell with recombinant DNA may be carried out by conventional techniques as are well known to those skilled in the art.

Where the host is prokaryotic, such as E. coli, competent cells which are capable of DNA uptake can be prepared from cells harvested after exponential growth phase and subsequently treated by the CaCl₂) method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransfected with DNA sequences encoding the fusion polypeptide of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukagotic Viral Vectors, Cold Spring Harbor Laboratory, Gluzman ed., 1982). Techniques for the isolation and purification of polypeptides of the invention expressed in prokaryotes or eukaryotes may be by any conventional means such as, for example, preparative chromatographic separations and immunological separations such as those involving the use of monoclonal or polyclonal antibodies or antigen.

It will be understood that the compounds of the present invention can be formulated in pharmaceutically and or diagnostically useful compositions. Such pharmaceutical and diagnostically useful compositions may be prepared according to well known methods. For example, MTS compounds having features of the invention, and having a cargo portion C that is, for example, a therapeutic moiety or a detection moiety, may be combined in admixture with a pharmaceutically acceptable carrier vehicle or a diagnostic buffering agent. Suitable vehicles and agents and their formulation, inclusive of other human proteins, e.g. human serum albumin are described, for example, in Remington's Pharmaceutical Sciences by E. W. Martin and, the techniques described in Maniatis, et al., Molecular Cloning A Laboratory Manual, Cold Spring Harbor Laboratory, N.Y., 1989-2013, which are hereby incorporated by reference. Such compositions will contain an effective amount of the compounds hereof together with a suitable amount of vehicle in order to prepare pharmaceutically acceptable compositions suitable for effective administration. Dosages and dosing regimens may be determined for the indications and compounds by methods known in the art, including determining (e.g., in experimental animals) the effective dose which causes half of those treated to respond to the treatment (ED₅₀) by providing a range of doses to experimental animals or subjects and noting the responses.

Sequence Listings:

| | Sequence |
|---|---|
| Cetuximab | QVQLKQSGPGLVQPSQSLSITCTVSGFSLTNYGVHWVRQSPGKGLEW |
| sequence | LGVIWSGGNTDYNTPFTSRLSINKDNSKSQVFFKMNSLQSNDTAIYYC |
| | ARALTYYDYEFAYWGQGTLVTVSAASTKGPSVFPLAPSSKSTSGGTA |
| | ALGCLVKDYFPEPVTVSWNSGALTSGVHTFPAVLQSSGLYSLSSVVTV |
| | PSSSLGTQTYICNVNHKPSNTKVDKRVEPKSCDKTHTCPPCPAPELLG |
| | GPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALP |
| | APIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSC |
| | SVMHEALHNHYTQKSLSLSPGK (SEQ ID NO: 1) |
| | DILLTQSPVILSVSPGERVSFSCRASQSIGTNIHWYQQRTNGSPRLLIK |
| | YASESISGIPSRFSGSGSGTDFTLSINSVESEDIADYYCQQNNNWPTTF |
| | GAGTKLELKRTVAAPSVFIFPPSDEQLKSGTASVVCLLNNFYPREAKV |
| | QWKVDNALQSGNSQESVTEQDSKDSTYSLSSTLTLSKADYEKHKVYA |
| | CEVTHQGLSSPVTKSFNRGEC (SEQ ID NO: 2) |
| Cetuximab | QVQLKQSGPGLVQPSQSLSITCTVSGFSLTNYGVHWVRQSPGKGLEW |
| Variable | LGVIWSGGNTDYNTPFTSRLSINKDNSKSQVFFKMNSLQSNDTAIYYC |
| heavy (vh) | ARALTYYDYEFAYWGQGTLVTVSAASTKGPSVFPLAPSSKSTSGGTA |
| domain | ALGCLVKDYFPEPVTVSWNSGALTSGVHTFPAVLQSSGLYSLSSVVTV |
| | PSSSLGTQTYICNVNHKPSNTKVDKRVEPKSCDKTHTCPPCPAPELLG |
| | GPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALP |
| | APIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSC |
| | SVMHEALHNHYTQKSLSLSPGK (SEQ ID NO: 1) |
| Cetuximab | FSLTNYGVH (SEQ ID NO: 3) |
| vhCDR1 | |
| Cetuximab | WSGGNTDYN (SEQ ID NO: 4) |
| vhCDR2 | |
| Cetuximab | ALTYYDYEFAY (SEQ ID NO: 5) |
| vhCDR3 | |
| Cetuximab | DILLTQSPVILSVSPGERVSFSCRASQSIGTNIHWYQQRTNGSPRLLIK |
| Variable light | YASESISGIPSRFSGSGSGTDFTLSINSVESEDIADYYCQQNNNWPTTF |
| (vl) domain | GAGTKLELKRTVAAPSVFIFPPSDEQLKSGTASVVCLLNNFYPREAKV |
| | QWKVDNALQSGNSQESVTEQDSKDSTYSLSSTLTLSKADYEKHKVYA |
| | CEVTHQGLSSPVTKSFNRGEC (SEQ ID NO: 2) |
| Cetuximab | RASQSIGTNIH (SEQ ID NO: 6) |
| vlCDR1 | |
| Cetuximab | YASESIS (SEQ ID NO: 7) |
| vlCDR2 | |
| Cetuximab | QQNNNWPTT (SEQ ID NO: 8) |
| vlCDR3 | |
| | Sequence |
| Trastuzumab | EVQLVESGGGLVQPGGSLRLSCAASGFNIKDTYIHWVRQAPGKGLE |
| sequence | WVARIYPTNGYTRYADSVKGRFTISADTSKNTAYLQMNSLRAEDT |
| | AVYYCSRWGGDGFYAMDYWGQGTLVTVSSASTKGPSVFPLAPSSK |
| | STSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYK |
| | CKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 9) |
| | DIQMTQSPSSLSASVGDRVTITCRASQDVNTAVAWYQQKPGKAPKL |
| | LIYSASFLYSGVPSRFSGSRSGTDFTLTISSLQPEDFATYYCQQHYTTP |
| | PTFGQGTKVEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC (SEQ ID NO: 10) |
| Trastuzumab | EVQLVESGGGLVQPGGSLRLSCAASGFNIKDTYIHWVRQAPGKGLE |
| Variable | WVARIYPTNGYTRYADSVKGRFTISADTSKNTAYLQMNSLRAEDT |
| heavy (vh) | AVYYCSRWGGDGFYAMDYWGQGTLVTVSSASTKGPSVFPLAPSSK |
| domain | STSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYK |
| | CKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 9) |
| Trastuzumab | FNIKDTYIH (SEQ ID NO: 11) |
| vhCDR1 | |
| Trastuzumab | RIYPTNGYTRYADSVKGRFTIS (SEQ ID NO: 12) |
| vhCDR2 | |
| Trastuzumab | WGGDGFYAMDY (SEQ ID NO: 13) |
| vhCDR3 | |
| Trastuzumab | DIQMTQSPSSLSASVGDRVTITCRASQDVNTAVAWYQQKPGKAPKL |
| Variable light | LIYSASFLYSGVPSRFSGSRSGTDFTLTISSLQPEDFATYYCQQHYTTP |
| (vl) domain | PTFGQGTKVEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC (SEQ ID NO: 10) |
| Trastuzumab | RASQDVNTAVA (SEQ ID NO: 14) |
| vlCDR1 | |
| Trastuzumab | YSASFLYS (SEQ ID NO: 15) |
| vlCDR2 | |
| Trastuzumab | QQHYTTPPT (SEQ ID NO: 16) |
| vlCDR3 | |
| | Sequence |
| Adalimumab | EVQLVESGGGLVQPGRSLRLSCAASGFTFDDYAMHWVRQAPGKGL |
| sequence | EWVSAITWNSGHIDYADSVEGRFTISRDNAKNSLYLDMNSLRAEDT |
| | AVYYCAKVSYLSTASSLDYWGQGTLVTVSSASTKGPSVFPLAPSSKS |
| | TSGGTAALGCLVKDYFPEPVTVSWNSGALTSGVHTFPAVLQSSGLYS |
| | LSSVVTVPSSSLGTQTYICNVNHKPSNTKVDKKI (SEQ ID NO: 17) |
| | DIQMTQSPSSLSASVGDRVTITCRASQGIRNYLAWYQQKPGKAPKLL |
| | IYAASTLQSGVPSRFSGSGSGTDFTLTISSLQPEDVATYYCQRYNRAP |
| | YTFGQGTKVEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGE (SEQ ID NO: 18) |
| Adalimumab | EVQLVESGGGLVQPGRSLRLSCAASGFTFDDYAMHWVRQAPGKGL |
| Variable | EWVSAITWNSGHIDYADSVEGRFTISRDNAKNSLYLQMNSLRAEDT |
| heavy (vh) | AVYYCAKVSYLSTASSLDYWGQGTLVTVSS (SEQ ID NO: 17) |
| domain | |
| Adalimumab | FTFDDYAMH (SEQ ID NO: 19) |
| vhCDR1 | |
| Adalimumab | AITWNSGHIDYADSVEGRFTIS (SEQ ID NO: 20) |
| vhCDR2 | |
| Adalimumab | VSYLSTASSLDY (SEQ ID NO: 21) |
| vhCDR3 | |
| Adalimumab | DIQMTQSPSSLSASVGDRVTITCRASQGIRNYLAWYQQKPGKAPKLL |
| Variable light | IYAASTLQSGVPSRFSGSGSGTDFTLTISSLQPEDVATYYCQRYNRAP |
| (vl) domain | YTFGQGTKVEIK (SEQ ID NO: 18) |
| Adalimumab | RASQGIRNYLA (SEQ ID NO: 22) |
| vlCDR1 | |
| Adalimumab | YAASTLQS (SEQ ID NO: 23) |
| vlCDR2 | |
| Adalimumab | QRYNRAPYT (SEQ ID NO: 24) |
| vlCDR3 | |
| | Sequence |
| Cetuximab complimentary | QGQSGQCISPRGCPDGPYVMY (SEQ ID NO: 25) |
| binding member | |
| Trastuzumab complimentary | GSGSGSQLGPYELWELSHGSGS (SEQ ID NO: 26) |
| binding member | |
| Trastuzumab complimentary | QVSHWVSGLAEGSFG (SEQ ID NO: 27) |
| binding member | |
| Trastuzumab complimentary | LSHTSGRVEGSVSLL (SEQ ID NO: 28) |
| binding member | |
| Adalimumab complimentary | HIHDDLLRYYGW (SEQ ID NO: 29) |
| binding member | |
| Matrix Metalloproteinase | PLGLAG (SEQ ID NO: 30) |
| cleavage substrate | |
| Matrix Metalloproteinase | PLGC(met)AG (SEQ ID NO: 31) |
| cleavage substrate | |
| Matrix Metalloproteinase | RS-(Cit)-G-(homoF)-YLY (SEQ ID NO: 32) |
| cleavage substrate | |
| Matrix Metalloproteinase | CRPAHLRDSG (SEQ ID NO: 33) |
| cleavage substrate | |
| Matrix Metalloproteinase | SLAYYTA (SEQ ID NO: 34) |
| cleavage substrate | |
| Matrix Metalloproteinase | NISDLTAG (SEQ ID NO: 35) |
| cleavage substrate | |
| Matrix Metalloproteinase | PPSSLRVT (SEQ ID NO: 36) |
| cleavage substrate | |
| Matrix Metalloproteinase | SGESLSNLTA (SEQ ID NO: 37) |
| cleavage substrate | |
| Matrix Metalloproteinase | RIGFLR (SEQ ID NO: 38) |
| cleavage substrate | |
| Matrix Metalloproteinase 2 | TLSE-LH (SEQ ID NO: 39) |
| cleavage substrate | |
| Matrix Metalloproteinase 2 | TIAHLA (SEQ ID NO: 40) |
| cleavage substrate | |
| Matrix Metalloproteinase 9 | SNPYK-Y (SEQ ID NO: 41) |
| cleavage substrate | |
| Matrix Metalloproteinase 9 | SNPKG-Y (SEQ ID NO: 42) |
| cleavage substrate | |
| Matrix Metalloproteinase 9 | SNPYG-Y (SEQ ID NO: 43) |
| cleavage substrate | |
| Matrix Metalloproteinase 14 | RSHP(Hfe)TLY (SEQ ID NO: 44) |
| cleavage substrate | |
| Matrix Metalloproteinase 14 | RSHG(Hfe)FLY (SEQ ID NO: 45) |
| cleavage substrate | |
| Cathepsin K cleavage substrate | KLRFSKQ (SEQ ID NO: 46) |
| Plasmin cleavage substrate | RLQLKL (SEQ ID NO: 47) |
| Urokinase Plasminogen | YGRAAA (SEQ ID NO: 48) |
| Activator cleavage substrate | |
| Urokinase Plasminogen | YGPRNR (SEQ ID NO: 49) |
| Activator cleavage substrate | |
| Thrombin cleavage substrate | DPRSFL (SEQ ID NO: 50) |
| Thrombin cleavage substrate | PPRSFL (SEQ ID NO: 51) |
| Thrombin cleavage substrate | TRPSFL (SEQ ID NO: 52) |
| Thrombin cleavage substrate | Norleucine-TPRSFL (SEQ ID NO: 53) |
| Elastase cleavage substrate | RLQLK(acetyl)L (SEQ ID NO: 54) |
| Elastase cleavage substrate | RLQLA(acetyl)L (SEQ ID NO: 55) |
| Chymase cleavage substrate | GVAYSGA (SEQ ID NO: 56) |

### EXAMPLES

### Example 1: General Example of Activatable Specific Binding Member Complexes

This example establishes the configuration of activatable specific binding member complex.

### General Description

In one aspect of the present invention, cetuximab (an antibody preparation widely used to treat metastatic colon and advanced or recurrent heads and neck cancer) toxicity can be modulated by the synthetic covalent attachment of a weak complementary binding member which is released after protease cleavage. The complementary binding member can be tethered to the antibody, or first specific binding member, through a complementary binding member/linker with a protease cleavage substrate that targets active protease(s) present in tumor microenvironment but not skin, permitting the “activatable” antibody to be efficacious without off target toxicity (FIG. 1). The activatable specific binding member complex (left) can be activated through cleavage by protease (right), thus forming an activated specific binding member.

### Reduced Antibody Side Effects Due to Off Target Activity

The present invention can be applied to any antibody therapeutic including but not limited to antibodies directed at immunotherapy targets, such as PDL-1 (pembrolizumab) or CTLA-4 (ipilimumab) which in addition to cetuximab are also are known to have significant off target toxicity limiting their use to select patients. Pembrolizumab and ipilimumab cause serious side effects throughout the body including intestines, (colitis and perforations), liver (hepatitis which can lead to liver failure), skin (severe rash), and nerve (damage that can lead to paralysis). The side effects are likely immune-mediated although a complete understanding of the cause and mechanism of these side effects is not well understood. Side effects can occur during treatment but can also be seen weeks or months after discontinuation of antibody therapy. Because of the severe side effects patients need to be closely monitored for symptoms of these adverse reactions throughout treatment.

Another application outside of cancer is protease activating antibody for multiple sclerosis, which is currently treated with a4 integrin targeted antibody natalizumab. Natalizumab was at one point pulled of the market because of an increased risk of contracting a rare brain infection, called progressive multifocal leukoencephalopathy (PML), which usually leads to death or severe disability. Natalizumab also shows significant liver toxicity and can cause severe allergic reactions. Because of these severe side effects natalizumab is only used to treat active MS which can be challenging to diagnose (typically done using patient current symptoms). Variants of natalizumab with a safer toxicity profile would be used more routinely in MS patients early in diagnosis.

Another class of antibody drugs that can have serious off target affects and limit use are antibody drug conjugates (ADCs). ADC are a rapidly expanded drug market which by their very nature (a toxic drug, conjugated to a targeting antibody) have an increased potential for negative side effects compared to antibody alone. To address these antibody related side effects, the present invention provides a platform technology that can be modified to modulate an antibody of interest. Cetuximab, which is currently used to treat several types of cancer in cancer of the head and neck, has been modified as Protease Activated cetuximab (PA-cetuximab). PA-cetuximab includes a synthetic CDR blocking domain attached through an extended PEG linker containing a protease cleavable substrate that is covalently linked to secondary sites on the antibody surface. The blocking domain is tethered to reactive lysines. The methodology of utilizing reactive lysines on the antibody surface allows the conjugation of multiple inhibitory domains that can contain protease activation domains for one or more disease selective protease(s). The number of reactive lysines on a giving antibody can vary but there is preferably between about 20 and about 30 fairly reactive sites(6) that can range in distance from about 5 to about 200 angstroms from the CDR antigen binding region. Covalent conjugation can alternatively be done with partial reduction of antibody and labeling of cysteine residues, as has been reported for the generation of drug conjugates. These modulating domains can be attached to specific substituted amino acids so that the exact number and location to the modulation function can be controlled. Attachment can be done using modified amino acids and click-chemistry or other more recently established methods that are being evaluated for the generation of antibody drug conjugates (REF). Cetuximab and a cyclic peptide inhibitory domain linked through an extended PEG (-100 angstroms) linker and protease cleavage site (Nle-TPRSFL) optimized for thrombin has been evaluated (7-9). The antibody modification of the present invention allows for dual targeting of the antibody to disease tissues were both antigen and protease(s) are present. Because this approach is modular it can be varied for application to any antibody, notably those that have off target toxicity including immunotherapy targets, such as PDL-1 or CTLA4-1.

Protease activated antibody could also be coupled to imaging agents to increase target selective labeling. Imaging agents could include fluorescent dye, PET agents, MRI agents or other contrast or imaging agent.

Notably, certain aspects of the present invention relate to the access to disease selective protease substrates. Certain protease selective Activatable Cell Penetrating Peptides (ACPPs) for selective targeting of imaging and therapeutic agents to cancer, multiple sclerosis, stroke, asthma, atherosclerosis and arthritis have been identified (4, 10-15) (Table 1). Identified substrates include novel sequences that are selectively cleaved by MMPs, uPA, elastase, chymase, plasmin, ADAMTS, and thrombin. Novel substrates for MMPs and uPA have recently been shown to be selectively cleaved in head and neck cancers (commonly treated with cetuximab). Protease activated antibody technology could also be combined with antibody drug conjugates (16).

TABLE 1
Table 1. Kinetic analysis of enzyme optimized RACPP with Kcat/Km measurement for selected enzymes.

| | Selectivity | MMP-2 | MMP-9 | MMP-12 | MMP-14 | Elastase | uPA | Thrombin | Plasmin | CathepsinK | Chymase |
|---|---|---|---|---|---|---|---|---|---|---|---|
| | MMPs | | | | | | | | | | |
| PLGC(me)AG | MMP2/9/ | 36429 | 13503 | 9167 | 17173 | 4001 | 1438 | — | — | 2640 | — |
| | 12/14 | | | | | | | | | | |
| TIAHLA | MMP2/ | 16243 | — | 2318 | 3349 | 18246 | — | — | — | 833 | 11508 |
| | Elastase | | | | | | | | | | |
| TLSLEH | MMP2 | 11405 | — | 1401 | 1200 | — | — | — | — | 497 | 2272 |
| RS(cit) | MMP14 | — | — | — | 4056 | — | — | — | — | — | — |
| G(Hfe)YLY | | | | | | | | | | | |
| PLGLEEA | MMP12 | 2417 | 1482 | 10459 | 1317 | — | — | — | — | 787 | — |
| SYPYKY | MMP12/ | — | — | 13952 | — | — | — | — | 19355 | — | 16361 |
| | Plasmin/ | | | | | | | | | | |
| | Chymase | | | | | | | | | | |
| | Serine | | | | | | | | | | |
| | Proteases | | | | | | | | | | |
| YGRAAA | Plasmin | 426 | — | — | — | 943 | 834 | — | 7716 | — | — |
| TGRAAA | uPA/Plasmin | — | — | — | — | 349 | 5578 | — | 4159 | — | — |
| RLQLK(Ac)L | Elastase | — | — | — | — | 13435 | — | — | — | 2873 | — |
| GVAYSGA | Chymase | — | — | — | — | 4547 | — | — | — | 1067 | 48133 |
| Nle-TPRSFL | Thrombin | — | — | — | — | — | — | 670000 | 13000 | 2353 | — |
| | Cysteine | | | | | | | | | | |
| | Proteases | | | | | | | | | | |
| KLRFSQK | Cathepsin/ | — | — | — | — | — | — | — | 6507 | 7439 | 1974 |
| | Plasmin | | | | | | | | | | |

We have used literature, rational design, and optimization and selection screens to identify and synthesize reasonably selective substrates and Cy5/Cy7 RACPPs for MMPs, Serine proteases and Cysteine proteases.

### Protease Activated Antibodies for Cancer

Cetuximab is still a go to treatment for head and neck carcinoma (HNC). HNC which includes cancers of the oral cavity, oropharynx and larynx is the 6^{th} most common cancer worldwide with an estimated annual burden of 355,000 deaths and 633,000 incident cases (17). Although the primary treated for HNC cancers is surgical resection (18) a recent review of the National Cancer Database of over 20,000 cases showed that the incidence of positive margins for surgery of the oral cavity ranges from 0-43.8% with an average of 7.5% (19). Option for secondary treatment include a second surgical resection with or without adjuvant ionizing radiation, or chemotherapy such as cetuximab.

There are multiple strategies based on antibodies against surface markers or ligands for receptors preferentially expressed in cancer (20). Extracellular proteases are mechanistically important in cancer, particularly in angiogenesis and metastasis (21). The present invention, in certain aspects, provides the combination of protease-selective substrates with antibody targeted therapy to improving treatment effectiveness and reduce treatment related side effects. Multiple proteases have been evaluated for their roles in cancer growth, invasion and metastasis, including matrix metalloproteinases (MMPs) (22) and urokinase plasminogen activator (uPA), cathepsins, interstitial collagenase (aka MMP1), elastases, (23), all of which can be used in the present invention to activate an activatable antibody by cleavage of a protease substrate localized in a linker, as an example.

MMPs are a class of endopeptidases that breakdown extracellular matrix leading to localized inflammation and tissue permeability both of which are associated with tumorigenesis and metastasis. Broad inhibition of MMPs for the treatment of advanced cancer has been unsuccessful in clinical trials (24). It is now recognized that MMPs can have both inhibitory and stimulatory effects on tumor progression (25, 26), thus a better understanding of the in vivo activity of specific MMPs in the context of cancer is needed to develop effective therapies or imaging agents. MMP2 and 9 are two very well studied gelatinases that can degrade collagen in the basement membrane which is postulated to be necessary for angiogenesis and metastasis (27). Also the inflammatory microenvironment within tumors causes upregulation of MMP2 and 9 via MMP14 activation leading to invasion in intestinal cancer (28). MMP14 (also known as MT1-MMP) is a membrane-tethered active protein that accumulate in invadopodia-like structures on the cell membrane to allow the cells to tunnel through the surrounding matrix (29). Inhibition of MMP14 expression with RNA interference had no effect on triple negative breast cancer cell growth but significantly diminished the number of migrating tumor cells and the incidence of lung metastasis (30).

Although MMP2,9 are also increased in inflammation/wound healing, absolute levels of these gelatinases in the head and neck have been used to differentiate between benign papillomas versus carcinoma of the larynx (31). Increased MMP2,9 expression has been shown to correlate with cancer grade (32) and decreased survival (33, 34). In carcinoma of the tongue, increased MMP2,9 expression has been shown to correlate with incidence of lymph node metastases (35). In addition to the well-studied role of MMPs, plasminogen activation is also believed to be important in the progression of multiple human cancers by facilitating matrix degradation during invasion and metastasis (36). Urokinase plasminogen activator (uPA) levels as measured by zymography has been shown be highly increased in tumor compared to adjacent normal tissue (23). From TCGA data analysis, it has been found that uPA mRNA expression is highly increased in tumor compared to paired normal tissue for multiple cancers including HNC.

### Example 2: Activatable Specific Binding Member Complexes that are Selectively Activated in Head and Neck Cancer (HNCC)

This example establishes the configuration of activatable cetuximab complex for targeting head and neck cancer.

Generate Activatable Cetuximab Complex that is Activated with HNCC Expressed MMPs and uPA.

A. Generate Activatable Cetuximab Complex

Synthesis and testing of uPA activatable cetuximab complex with NHS or Maleimide (for covalent linkage), PEG linker, MMP (PLGCmetAG) and uPA substrate (TGRAAA), and cyclic cetuximab binding peptide to make activatable cetuximab complex of configuration (Acety-QGQSGQCISPRGCPDGPYVMY-PEG6-(Nle-TPRSFL)-PEG24)p-cetuximab), (Acety-QGQSGQCISPRGCPDGPYVMY-PEG6-(PLGCmetAG)-PEG24)p-cetuximab) and (Acety-QGQSGQCISPRGCPDGPYVMY-PEG6-(TGRAAA)-PEG24)p-cetuximab.

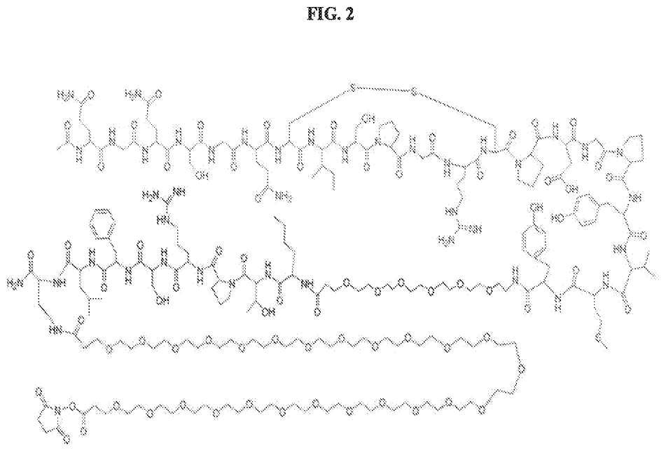

Synthetically altered cetuximab (sourced from UCSD Moores cancer pharmacy) by covalent attachment of a complementary binding member/linker as exemplified in (FIG. 2) which contains a reactive handle (NHS ester), a flexible 90 angstrom linker (PEG24), a thrombin cleavable substrate (Nle-TPRSFL) and an complementary binding member that blocks cetuximab binding to EGFR.

Synthesis of Activatable Cetuximab Complex with Cetuximab (FIG. 1) Covalently Linked to Complementary Binding Member/Linker

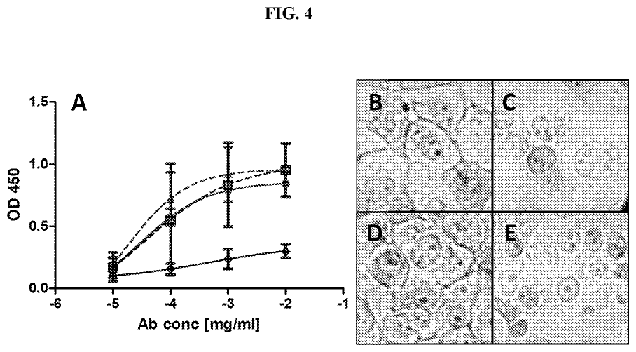

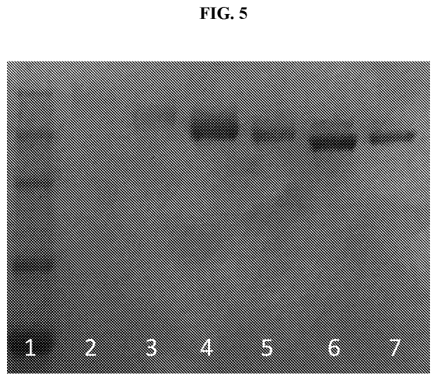

Peptide with structure acetyl-QGQSGQCISPRGCPDGPYVMY-PEG6-(Nle)TPRSFL-(diamino proprionic)-amide was synthesized using standard solid phase Fmoc synthesis using a Prelude synthesizer (Protein Technologies Inc). Peptides were purified using C-18 reverse-phase HPLC and characterized using analytical HPLC, combined with mass spectrometry and confirmed to be >90% purity. Purified peptide was then reacted with 2 equivalents Bis-dPEG₂₅-NHS ester (Quanta Biodesign Cat #10968) in dimethyl-sulfoxides with N-methylmorpholine. Peptide-PEG24 conjugated was purified using C-18 reverse-phase HPLC and characterized using analytical HPLC, combined with mass spectrometry and confirmed to be >90% purity. Peptide-PEG conjugate of structure acetyl-QGQSGQCISPRGCPDGPYVMY-peg-NleTPRSFL-(diamino proprionic)-PEG24-NHS (FIG. 2) was then reacted at 10, 50 and 100 equivalents in terms of molar ratio of peptide-PEG conjugate to cetuximab antibody. Reactions were done with 1.8 mg/ml cetuximab (sourced from UCSD pharmacy) in 100 mM bicine buffer pH 8.3 with addition of peptide-PEG-NHS conjugate at fixed molar excess (10, 50 and 100 equivalents). Excess NHS was neutralized with addition of ethanolamine. Conjugated-cetuximab was either used directly after labeling or purified using size exclusion column to remove any excess unreacted peptide-PEG-NHS. Cetuximab could be additionally be labeled with fluorophore by addition of 5 to 10 percent dye NHS in the Peptide-PEG conjugate/cetuximab reaction. Conjugation of cetuximab to PEG-peptide conjugate was confirmed by gel functional blocking (FIG. 4) and gel electrophoresis (FIG. 5). We estimate that 4 to 30 peptide-PEG conjugates are covalently linked to cetuximab based on size modification. The number of reactive lysines on a giving antibody can vary but there are 20 to 30 reactive lysines (Gautier et. al. Proteomic. 2015) that range in distance (˜5 to 200 angstroms) from the CDR antigen binding region.

Activatable cetuximab complex is generated with substrates that are selectively cleaved in head and neck cancer. Activatable cetuximab complex are synthesized using the 4 protease substrates that have been shown to target HSNCC using RACPPS. Activatable cetuximab complexes with the configurations of (Acety-QGQSGQCISPRGCPDGPYVMY-Peg6-PLGC(met)AG-Peg24)\-cetuximab), (Acety-QGQSGQCISPRGCPDGPYVMY-Peg6-YGRAAA-Peg24-cetuximab, (Acety-QGQSGQCISPRGCPDGPYVMY-Peg6-TGRAAA-Peg24-cetuximab and (Acety-QGQSGQCISPRGCPDGPYVMY-Peg6-KLRFSQK-Peg24-cetuximab are synthesized and tested in-vitro. Synthesis of activatable cetuximab complexes can be done with modular parts so that the protease substrate can be easily swapped during parallel synthesis.

Rational and empirical strategies have been used to develop a panel of new RACPPs that are highly selective for and efficiently cleaved by MMP2, MMP12, MMP14, urokinase plasminogen activator, elastase, plasmin, thrombin, cathepsins and chymase. Differential tumor targeted of each RAAPP with be evaluate in-vitro fallowed by in-vivo comparison with alternative targeting strategies including antibody/nanobody directed agents.

Using literature, rational design and optimization and selection screens to identify reasonably selective ACPP substrates for MMP14, MMP-2, MMP12, urokinase plasminogen activator, thrombin, elastase, cathepsin, chymase, and plasmin (TABLE 1).

### Protease Activated Antibodies for Cancer

Cetuximab is still a go to treatment for head and neck carcinoma (HNC). HNC which includes cancers of the oral cavity, oropharynx and larynx is the 6^{th} most common cancer worldwide with an estimated annual burden of 355,000 deaths and 633,000 incident cases (17). Although the primary treated for HNC cancers is surgical resection (18) a recent review of the National Cancer Database and over 20,000 cases showed that the incidence of positive margins for surgery of the oral cavity ranges from 0-43.8% with an average of 7.5% (19). Option for secondary treatment include a second surgical resection with or without adjuvant ionizing radiation, or chemotherapy such as cetuximab.

In addition to cetuximab there are multiple other strategies based on antibodies against surface markers or ligands for receptors preferentially expressed in cancer that could be targeted with this methodology (20). Extracellular proteases are also known to mechanistically important in cancer, particularly in angiogenesis and metastasis (21) which are sometimes over-expressed in cancer.

One aspect of the present invention is to combine newly identified protease-selective substrates (TABLE 1) with antibody targeted therapy to improve treatment effectiveness and reduce treatment related side effects of antibody therapy. Many proteases have been evaluated for their roles in cancer growth, invasion and metastasis, including matrix metalloproteinases (MMPs) (22) and urokinase plasminogen activator (uPA), cathepsins, interstitial collagenase (aka MMP1), elastases (23), and others. In carcinoma of the tongue, increased MMP2,9 expression has been shown to correlate with high incidence of lymph node metastases (35). MMPs and plasminogen activators are also believed to be critical in the progression of multiple human cancers by facilitating matrix degradation during invasion and metastasis (36). Urokinase plasminogen activator (levels as measured by zymography has been shown be highly increased in tumor compared to adjacent normal tissue (23). Substrates selective for MMPs, plasmin, uPA and cathepsin have shown particular interest for coupling to protease activated cetuximab as protease selective RACPPs show specific uptake in head and neck cancer (FIG. 2).

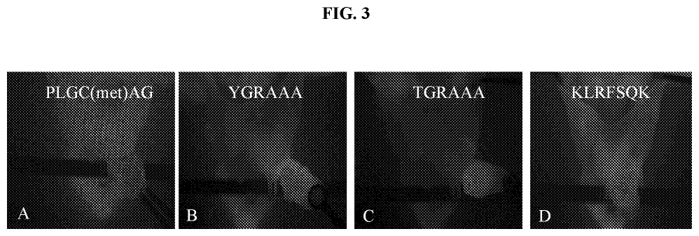

In these results, ratiometric fluorescence was assessed in mice bearing orthotopic Cal-27 xenograft tumors, a model for head and neck carcinoma, following IV injection of ratiometric ACPPs (RACPPs) with varying cleavage sequences. Our traditional PLG⬇C(Me)AG RACPP(37) (FIG. 3A) gave good cleavage in the tumor but also had high signal in adjacent tongue. The plasmin/uPA-cleavable probe (YGR⬇AAA) showed tumor specific labeling at high intensity with very low labeling of adjacent tissue (FIG. 3B), uPA selective (TGR⬇AAA) showed high level localized uptake at the center of the tumor (FIG. 2C). Surprisingly a cathepsin cleavable probe with substrate (KLR⬇FSQK) also gave very selective targeting to cancerous tongue (FIG. 3D). FIG. 2. In vivo ratiometric fluorescence imaging of RACPPs in orthotopic tongue tumor model (Cal27) for protease selective probes. Tongues are restrained and imaged as shown in FIG. 3.

### Example 3: Generation of Activatable Specific Binding Member Complex

This example establishes the generation of an activatable specific binding member complex that is activated by protease pro-coagulation enzymes.

### Protease Activation of Cetuximab by Thrombin Results in Selective Binding to EGFR

Synthesis and testing of uPA activatable cetuximab with NHS or Maleimide (for covalent linkage), PEG linker, MMP (PLGCmetAG) and uPA substrate (TGRAAA), and cyclic cetuximab binding peptide to make PA-cetuximab of configuration (Acety-QGQSGQCISPRGCPDGPYVMY-PEG6-(Nle-TPRSFL)-PEG24)ₙ-cetuximab), (Acety-QGQSGQCISPRGCPDGPYVMY-PEG6-(PLGCmetAG)-PEG24)ₙ-cetuximab) and (Acety-QGQSGQCISPRGCPDGPYVMY-PEG6-(TGRAAA)-PEG24)ₙ-cetuximab.

Synthetically altered cetuximab (sourced from UCSD Moores cancer pharmacy) by covalent attachment of a synthetic peptide biomolecule as exemplified in (FIG. 2) which contains a reactive handle (NHS ester), a flexible 90 angstrom linker (PEG24), a thrombin cleavable substrate (Nle-TPRSFL) and a complementary binding member that blocks cetuximab binding to EGFR.

Data: Synthetically altered cetuximab (sourced from UCSD Moores cancer pharmacy) by covalent attachment of a synthetic peptide biomolecule (FIG. 2) which contains a reactive handle (NHS ester), a flexible 90 angstrom linker (PEG24), a thrombin cleavable activation domain (Nle-TPRSFL) and a complementary binding member that blocks cetuximab binding to EGFR. Thrombin treatment activated the PA-cetuximab to allow binding to EGFR protein and cells (Cal-27) that express EGFR (FIG. 4).

Thrombin activation of PA-cetuximab cause a size shift in inhibited complex that can be detected after running on acrylamide gel. Protease cleavage of blocked PA-cetuximab causes size change as CDR blocking domains is proteolytically released from cetuximab panibody. Lane 1 is a protein marker. Lane 2 cetuximab conjugated to high level of protease releasable CDR blocking domain. Lane 3 cetuximab conjugated to high level of protease releasable CDR blocking domain after treatment with thrombin. Lane 4 cetuximab conjugated to low level of protease releasable CDR blocking domain. Lane 5 cetuximab conjugated to low level of protease releasable CDR blocking domain after treatment with thrombin. Lane 6 and 7 are control unmodified cetuximab before (lane 6) and after treatment with thrombin (lane 7) (FIG. 5)

### Example 4: Cetuximab Binding to EGFR can be Sterically Blocked by Synthetic Attachment of Large Polyethlene Glycols

This example establishes the use of large steric groups that can be attached to antibody and inhibit binding to target until large steric group is released by protease

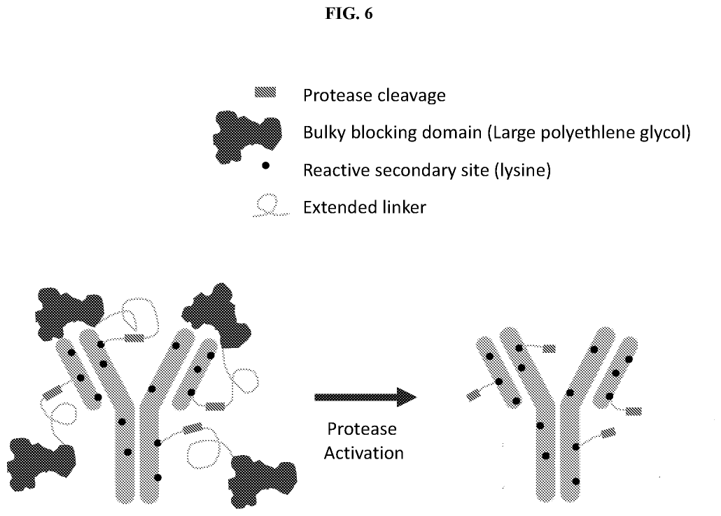

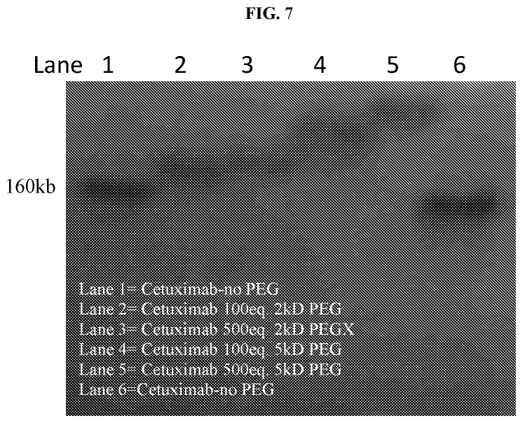

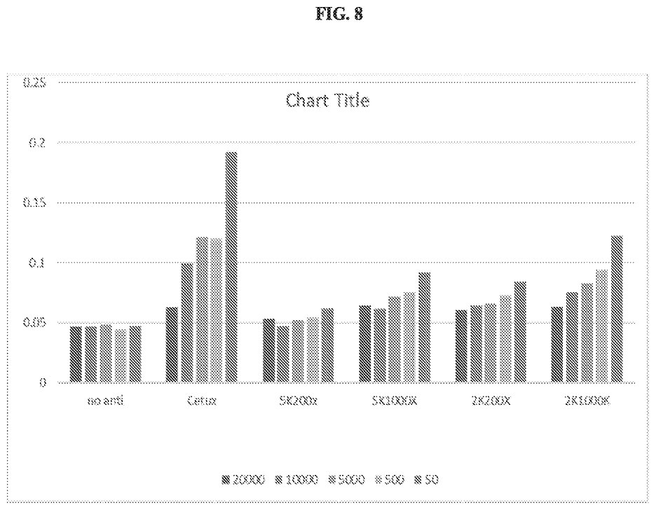

Generation of Protease Activatable Antibody by synthetic attachment of a bulky molecule with a protease cleavable linker. Schematic shows attachment of bulky peq groups to lysine residue on antibody using NHS-ester (FIG. 6). PEG conjugation blocks cetuximab binding to target receptor EGFR. High protein binding 96 well plates were coated with 20 ul EGFR in 0.2 M Sodium Bicarbonate pH 9.6 overnight at 4 degrees C. Plates were then washed 3 times with PBS and blocked overnight at 4 degrees with degrees with PBS and 0.5% BSA. Control antibody and pegylated antibodies were added at dilution from 1 to 50 to 20K PEG/antibody in PBS with 0.5% BSA. Cetuximab antibody was prelabeled with either 100 or 500 equivalents of 5K PEG-NHS causing a size shifts by gel electrophoresis as shown in FIG. 7. There are 2 binding sites per antibody so 100 equivalents is equal to 200× and 500 equivalents is equal 100×. Antibody was incubated on plates for 24 hours at 4 degrees C. and then decanted off. Plates were washed 5 times with PBST. Secondary antibody (Goat anti Human) conjugated to HRP was added at a dilution of 1 to 1000 in PBS with 0.5% BSA. Plates were incubated oat RT for 3 hours followed by washing 5 time with PBST. One step TMB was added and plates were incubated at RT for ˜1 hour prior to imaging as shown. Graph shown highest binding inhibition with 5K PEG with 200× crosslinked inhibitor (FIG. 8).

### Example 5. Generation of Reactive Peptide Inhibitor Complex (Pic) for Covalent Linkage to Cetuximab

Peptide with structure acetyl-QGQSGQCISPRGCPDGPYVMY-PEG6-(Nle)TPRSFL-(diamino proprionic (Dap))-amide was synthesized using standard solid phase Fmoc synthesis using a Prelude synthesizer (Protein Technologies Inc). Peptides were purified using C-18 reverse-phase HPLC and characterized using analytical HPLC, combined with mass spectrometry and confirmed to be >90% purity.

A) To generate amine reactive PIC, purified peptide was then reacted with 2 equivalents Bis-dPEG₂₅-NHS ester (Quanta Biodesign Cat #10968) in dimethyl-sulfoxides with N-methylmorpholine. Peptide-PEG24 conjugate with structure acetyl-QGQSGQCISPRGCPDGPYVMY-peg-NleTPRSFL-(diamino proprionic)-PEG24-NHS was purified using C-18 reverse-phase HPLC and characterized using analytical HPLC, combined with mass spectrometry and confirmed to be >90% purity.

B) To generate thiol reactive PIC purified peptide was then reacted with 2 equivalents Maleimide-dPEG₂₅-NHS ester (Quanta Biodesign) in dimethyl-sulfoxides with N-methylmorpholine to form PIC with structure acetyl-QGQSGQCISPRGCPDGPYVMY-peg-NleTPRSFL-(diamino proprionic (Dap))-PEG24-maleimide. Peptide-PEG24 conjugate with structure acetyl-QGQSGQCISPRGCPDGPYVMY-peg-NleTPRSFL-(diamino proprionic)-PEG24-maleimide was purified using C-18 reverse-phase HPLC and characterized using analytical HPLC, combined with mass spectrometry and confirmed to be >90% purity.

For synthesis of uPA or MMP targeted PIC peptides with the structures below were used with analogous method from above, MMP (PLGCmetAG) and uPA substrate (TGRAAA). (Acety-QGQSGQCISPRGCPDGPYVMY-PEG6-(PLGCmetAG)-PEG24)-Dap) and (Acety-QGQSGQCISPRGCPDGPYVMY-PEG6-(TGRAAA)-PEG24)-Dap.

### Example 6. Generation of Reactive Peptide Inhibitor Complex (Pic) for Covalent Linkage to Trastuzumab

Three trastuzumab inhibitor peptides were synthesized with sequences: 1) GSGSGSQLGPYELWELSHGSGS; 2) QVSHWVSGLAEGSFG; and 3) LSHTSGRVEGSVSLL. Peptide with structure acetyl-(GSGSGSQLGPYELWELSHGSGS)-PEG6-(Nle)TPRSFL-(diamino proprionic (Dap))-amide was synthesized using standard solid phase Fmoc synthesis using a Prelude synthesizer (Protein Technologies Inc). Peptides were purified using C-18 reverse-phase HPLC and characterized using analytical HPLC, combined with mass spectrometry and confirmed to be >90% purity.

A) To generate amine reactive PIC purified peptide was then reacted with 2 equivalents Bis-dPEG₂₅-NHS ester (Quanta Biodesign Cat #10968) in dimethyl-sulfoxides with N-methylmorpholine. Peptide-PEG24 conjugate with structure acetyl-GSGSGSQLGPYELWELSHGSGS-PEG-NleTPRSFL-(diamino proprionic)-PEG24-NHS was purified using C-18 reverse-phase HPLC and characterized using analytical HPLC, combined with mass spectrometry and confirmed to be >90% purity.

B) To generate thiol reactive PIC purified peptide was then reacted with 2 equivalents Maleimide-dPEG₂₅-NHS ester (Quanta Biodesign) in dimethyl-sulfoxides with N-methylmorpholine to form PIC with structure acetyl-GSGSGSQLGPYELWELSHGSGS-PEG-NleTPRSFL-(diamino proprionic (Dap))-PEG24-maleimide. Peptide-PEG24 conjugate with structure acetyl-GSGSGSQLGPYELWELSHGSGS-PEG-NleTPRSFL-(diamino proprionic)-PEG24-maleimide was purified using C-18 reverse-phase HPLC and characterized using analytical HPLC, combined with mass spectrometry and confirmed to be >90% purity.

### Example 7. Generation of Reactive Peptide Inhibitor Complex (Pic) for Covalent Linkage to Adalimumab

Peptide with structure acetyl-(HIHDDLLRYYGW)-PEG6-(Nle)TPRSFL-(diamino proprionic (Dap))-amide was synthesized using standard solid phase Fmoc synthesis using a Prelude synthesizer (Protein Technologies Inc). Peptides were purified using C-18 reverse-phase HPLC and characterized using analytical HPLC, combined with mass spectrometry and confirmed to be >90% purity.

A) To generate amine reactive PIC purified peptide was then reacted with 2 equivalents Bis-dPEG₂₅-NHS ester (Quanta Biodesign Cat #10968) in dimethyl-sulfoxides with N-methylmorpholine. Peptide-PEG24 conjugate with structure acetyl-HIHDDLLRYYGW-peg-NleTPRSFL-(diamino proprionic)-PEG24-NHS was purified using C-18 reverse-phase HPLC and characterized using analytical HPLC, combined with mass spectrometry and confirmed to be >90% purity.

B) To generate thiol reactive PIC purified peptide was then reacted with 2 equivalents Maleimide-dPEG₂₅-NHS ester (Quanta Biodesign) in dimethyl-sulfoxides with N-methylmorpholine to form PIC with structure acetyl-HIHDDLLRYYGW-peg-NleTPRSFL-(diamino proprionic (Dap))-PEG24-maleimide. Peptide-PEG24 conjugate with structure acetyl-HIHDDLLRYYGW-peg-NleTPRSFL-(diamino proprionic)-PEG24-maleimide was purified using C-18 reverse-phase HPLC and characterized using analytical HPLC, combined with mass spectrometry and confirmed to be >90% purity.

For synthesis of uPA or MMP, cathepsin targeted PIC peptides with the structures below were used with analogous method from above, MMP (PLGCmetAG) and uPA substrate (TGRAAA). MMP; (Acety-HIHDDLLRYYGW-PEG6-(PLGCmetAG)-PEG24)-Dap) and UpA; (Acety-HIHDDLLRYYGW-PEG6-(TGRAAA)-PEG24)-Dap, cathepsin; (Acety-HIHDDLLRYYGW-PEG6-(KLRFSQK)-PEG24)-Dap.

### Example 8. Generation of Protease Activatable Antibodies by Synthetic Conjugation of Peptide-Inhibitor Complex to Reactive Amines (Lysines, N-Termini)

A solution (1 ml, 2 mg ml⁻¹) of antibody i.e. [cetuximab (Erbitux, ImClone) or trastuzumab (Herceptin, Roche), adlimumab (Humira, Abbvie)] was treated with sodium bicine buffer (100 μl, 1 M pH 8.3). Antibody solution was then added to varied equvilents (1× to 1000× equivalents) of Peptide Inhibitor Complex PIC-NHS (See Example 5A for cetuximab; See Example 6A for trastuzumab; See Example 7A for adlimumab) ester conjugate conjugated (to react with exposed lysines or N-termini on the antibody) followed by 24 hour incubation at room temperature. The number of reactive lysines on a giving antibody can vary but there is preferably between about 20 and about 30 fairly reactive sites(6) that can range in distance from about 5 to about 200 angstroms from the CDR antigen binding region.

To additionally label with Cy5 using Cy5-NHS-ester (2 equivalents of Cy5 NHS) was added followed by incubation for an additional 24 hours. Antibody-PIC conjugate was then gel-filtered (Sephadex G25, 0.6 g) to remove excess PIC and Cy5 and eluted with PBS. Following centrifugal concentration (Centricon 30 kDa MWCO) to 500 μl, the concentrations of antibody and Cy5 were determined by absorbance using extinction coefficients of 210,000 M⁻¹ cm⁻¹ (cetuximab) or 225,000 M⁻¹ cm⁻¹ (trastuzumab) at 280 nm and 12,500 M⁻¹ cm⁻¹ and 250,000 M⁻¹ cm⁻¹ at 280 and 650 nm, respectively, for Cy5. Peptide conjugation was measured by denaturing reverse-phase HPLC of the reaction mix before addition of Cy5 NHS, following reduction of disulfides with 50 mM DTT for 30 min. Peaks corresponding to light or heavy chains with 0-50 PICs were identified by electro-spray mass spectroscopy and peak areas at 280 nm were integrated and weighted to calculate the drug loading.

### Example 9. Generation of Protease Activatable Antibodies by Synthetic Conjugation of Peptide-Inhibitor Complex to Reduced Cysteines

A solution (1 ml, 2 mg ml⁻¹) of antibody i.e. [cetuximab (Erbitux, ImClone) or trastuzumab (Herceptin, Roche) or adlimumab (Humira, Abbvie)] was treated with sodium bicine buffer (100 μl, 1 M pH 8.3) and sodium diethylenetriaminepentaacetic acid (10 μl, 100 mM pH 7). Following reduction with four equivalents of tris(carboxyethyl)phosphine (TCEP) at 37° C. for 2 h, the solution was added to four equivalents of Peptide Inhibitor Complex (See Example 5B for cetuximab; See Example 6B for trastuzumab; See Example 7B for adlimumab) conjugated to Maleimide followed by incubation at room temperature for 30 min. To additionally label with Cy5 using Cy5-maleimide (2 equivalents of Cy5 maleimide) was added followed by incubation for an additional 30 min. Antibody-PIC conjugate was then gel-filtered (Sephadex G25, 0.6 g) to remove excess PIC and Cy5 and eluting with PBS. Following centrifugal concentration (Centricon 30 kDa MWCO) to 500 μl, the concentrations of antibody and Cy5 were determined by absorbance using extinction coefficients of 210,000 M⁻¹ cm⁻¹ (cetuximab) or 225,000 M⁻¹ cm⁻¹ (trastuzumab) at 280 nm and 12,500 M⁻¹ cm⁻¹ and 250,000 M⁻¹ cm⁻¹ at 280 and 650 nm, respectively, for Cy5. Peptide conjugation was measured by denaturing reverse-phase HPLC of the reaction mix before addition of Cy5 maleimide, following reduction of any remaining intersubunit disulfides with 50 mM DTT for 30 min. Peaks corresponding to light or heavy chains with 0-3 peptides were identified by electro-spray mass spectroscopy and peak areas at 280 nm were integrated and weighted to calculate the drug loading.

### Example 10. Generation of Protease Activatable Antibodies by Site Specific Synthetic Conjugation of Peptide-Inhibitor Complex to Substituted Amino Acids

Antibodies can be genetically modified with amino acids including glutamine or non-native amino acids that have reactive sides chains including a ketone, azide, alkyne, alkene, and/or tetrazine side group.

Lysine to glutamine (which can be substituted) conjugation with microbial Tgase: For the conjugation of C16-HC and C16-LC to AcLys-vcMMAD, antibody was adjusted to 5 mg/mL in buffer containing 25 mM Tris-HCl at pH 8.0, and 150 mM NaCl, AcLys-vc-MMAD was added in either a 5-fold (C16-HC) or 10-fold (C16-LC) molar excess over antibody and the enzymatic reaction initiated by addition of 1% (w/v) (C16-HC) or 2% (w/v) (C16-LC) bacterial transglutaminase (Ajinomoto Activa TI, Japan). Following incubation with gentle shaking at 22° C. (C16-HC) or 37° C. (C16-LC) for 16 hours, the ADC was purified using MabSelect SuRe (GE Healthcare, Inc) using standard procedures.

Oxime ligation, alkoxyamine-to-keto-group reaction: Non native keto group containing mAb was conjugated to drug/linker under the following conditions: 10 mg mAb/mL, 10:1 drug/mAb molar ratio, 1% acetic hydrazide. Reaction incubated at 28° C. for 40-60 h. After incubation, conjugation reaction was diluted into 20 mM Tris, 0.75 M ammonium sulfate, pH 7, and loaded onto a Phenyl HP column (GE Healthcare) equilibrated in the same buffer. mAb was eluted from the column with a 0-100% linear gradient over 50 CV. Eluent buffer contained the following: 20 mM Tris, 20% isopropanol, pH 7.

Copper free click chemistry: The Trastuzumab variants were conjugated to an exemplary cytotoxic agent, MMAF, using a constrained cyclooctyne reagent. In brief, DBCO-PEGMMAF (ACME Bioscience; Palo Alto, Calif.) was dissolved in DMSO to a final concentration of 5 mM. The compound was diluted with PBS to 1 mM and then added to the purified protein sample in IMAC elution buffer to final drug concentration of 100 μM and a final pAMF-incorporated IgG concentration of 10 μM (10:1 molar ratio of drug-linker:IgG). This mixture was incubated at RT (25° C.) for 16 h. Reaction was stopped by adding sodium azide to final concentration of 1 mm and buffer exchanged using zeba plates (Thermo Scientific) equilibrated in 1×PBS. Filtrate was then passed through a MUSTANG Q plate (Pall Corp.) to remove endotoxin.

Hydrazine-to-aldehyde-group reaction: Aldehyde-tagged antibodies (15 mg/mL) were conjugated to HIPS-Glu-PEG2-maytansine (8 mol equiv drug:antibody) for 72 h at 37° C. in 50 mM sodium citrate, 50 mM NaCl pH 5.5 containing 0.85% DMA and 0.085% Triton X-100.

Free drug was removed using tangential flow filtration. Unconjugated antibody was removed using preparative-scale hydrophobic interaction chromatography (HIC; GE Healthcare 17-5195-01) with mobile phase A: 1.0 M ammonium sulfate, 25 mM sodium phosphate pH 7.0, and mobile phase B: 25% isopropanol, 18.75 mM sodium phosphate pH 7.0. An isocratic gradient of 33% B was used to elute unconjugated material, followed by a linear gradient of 41-95% B to elute mono- and diconjugated species.

Curr Opin Chem Biol. 2013 June; 17(3): 412-419. Tian, Feng, et al. “A general approach to site-specific antibody drug conjugates.” Proceedings of the National Academy of Sciences 111.5 (2014): 1766-1771. J. Biol. Chem. (2017) 292(38) 15622-15635; J Am Chem Soc. 2006 Apr. 12; 128(14): 4542-4543. doi:10.1021/ja0604111; Antibodies 2018, 7, 4; doi:10.3390/antib7010004; Zimmerman, Erik S., et al. “Production of site-specific antibody-drug conjugates using optimized non-natural amino acids in a cell-free expression system.” Bioconjugate chemistry 25.2 (2014): 351-361; Strop, Pavel, et al. “Location matters: site of conjugation modulates stability and pharmacokinetics of antibody drug conjugates.” Chemistry & biology 20.2 (2013): 161-167; Lysine to glutamine conjugation with microbial Tgase; Drake, Penelope M., et al. “Aldehyde tag coupled with HIPS chemistry enables the production of ADCs conjugated site-specifically to different antibody regions with distinct in vivo efficacy and PK outcomes.” Bioconjugate chemistry 25.7 (2014): 1331-1341.

### REFERENCES

A) https://www.grandviewresearch.com/press-release/global-monoclonal-antibodies-market https://www.ncbi.nlm.nih.gov/pmc/articles/PMC4622599/
B) https://news.bms.com/press-release/partnering-news/bristol-myers-squibb-and-cytomx-therapeutics-extend-worldwide-collabor https://www.genengnews.com/gen-news-highlights/bms-pays-cytomx-200m-upfront-to-expand-probody-therapeutics-partnership/81254050
1. Desnoyers L R, Vasiljeva O, Richardson J H, Yang A, Menendez E E, Liang T W, et al. Tumor-specific activation of an EGFR-targeting probody enhances therapeutic index. Science translational medicine. 2013; 5(207):207ra144. Epub 2013/10/18. doi: 10.1126/scitranslmed.3006682. PubMed PMID: 24132639.
2. Polu K R, Lowman H B. Probody therapeutics for targeting antibodies to diseased tissue. Expert opinion on biological therapy. 2014; 14(8):1049-53. Epub 2014/05/23. doi: 10.1517/14712598.2014.920814. PubMed PMID: 24845630.
3. Wong K R, Menendez E, Craik C S, Kavanaugh W M, Vasiljeva O. In vivo imaging of protease activity by Probody therapeutic activation. Biochimie. 2016; 122:62-7. Epub 2015/11/08. doi: 10.1016/j.biochi.2015.11.003. PubMed PMID: 26546838; PubMed Central PMCID: PMC5709043.
4. Chen B, Friedman B, Whitney M A, Winkle J A, Lei I F, Olson E S, et al. Thrombin activity associated with neuronal damage during acute focal ischemia. The Journal of neuroscience: the official journal of the Society for Neuroscience. 2012; 32(22):7622-31. Epub 2012/06/01. doi: 10.1523/JNEUROSCI.0369-12.2012. PubMed PMID: 22649241; PubMed Central PMCID: PMC3383068.
5. Donaldson J M, Kari C, Fragoso R C, Rodeck U, Williams J C. Design and development of masked therapeutic antibodies to limit off-target effects: application to anti-EGFR antibodies. Cancer biology & therapy. 2009; 8(22):2147-52. Epub 2009/09/29. PubMed PMID: 19783899; PubMed Central PMCID: PMC3546534.
6. Gautier V, Boumeester A J, Lossl P, Heck A J. Lysine conjugation properties in human IgGs studied by integrating high-resolution native mass spectrometry and bottom-up proteomics. Proteomics. 2015; 15(16):2756-65. Epub 2015/02/03. doi: 10.1002/pmic.201400462. PubMed PMID: 25641908.
7. Whitney M, Savariar E N, Friedman B, Levin R A, Crisp J L, Glasgow H L, et al. Ratiometric activatable cell-penetrating peptides provide rapid in vivo readout of thrombin activation. Angewandte Chemie. 2013; 52(1):325-30. Epub 2012/10/20. doi: 10.1002/anie.201205721. PubMed PMID: 23080482; PubMed Central PMCID: PMC3694763.
8. Olson E S, Whitney M A, Friedman B, Aguilera T A, Crisp J L, Baik F M, et al. In vivo fluorescence imaging of atherosclerotic plaques with activatable cell-penetrating peptides targeting thrombin activity. Integrative biology: quantitative biosciences from nano to macro. 2012; 4(6):595-605. Epub 2012/04/27. doi: 10.1039/c2ib00161f. PubMed PMID: 22534729; PubMed Central PMCID: PMC3689578.
9. Friedman B, Whitney M A, Savariar E N, Caneda C, Steinbach P, Xiong Q, et al. Detection of Subclinical Arthritis in Mice by a Thrombin Receptor-Derived Imaging Agent. Arthritis & rheumatology. 2017. Epub 2017/11/23. doi: 10.1002/art.40316. PubMed PMID: 29164814.
10. Whitney M, Crisp J L, Olson E S, Aguilera T A, Gross L A, Ellies L G, et al. Parallel in vivo and in vitro selection using phage display identifies protease-dependent tumor-targeting peptides. The Journal of biological chemistry. 2010; 285(29):22532-41. Epub 2010/05/13. doi: 10.1074/jbc.M110.138297. PubMed PMID: 20460372; PubMed Central PMCID: PMC2903386.
11. Raju S C, Hauff S J, Lemieux A J, Orosco R K, Gross A M, Nguyen L T, et al. Combined TP53 mutation/3p loss correlates with decreased radiosensitivity and increased matrix-metalloproteinase activity in head and neck carcinoma. Oral oncology. 2015; 51(5):470-5. Epub 2015/03/05. doi: 10.1016/j.oraloncology.2015.01.014. PubMed PMID: 25735654; PubMed Central PMCID: PMC4427339.
12. Hua N, Baik F, Pham T, Phinikaridou A, Giordano N, Friedman B, et al. Identification of High-Risk Plaques by MRI and Fluorescence Imaging in a Rabbit Model of Atherothrombosis. PloS one. 2015; 10(10):e0139833. Epub 2015/10/09. doi: 10.1371/journal.pone.0139833. PubMed PMID: 26448434; PubMed Central PMCID: PMC4598148.
13. Hauff S J, Raju S C, Orosco R K, Gross A M, Diaz-Perez J A, Savariar E, et al. Matrix-metalloproteinases in head and neck carcinoma-cancer genome atlas analysis and fluorescence imaging in mice. Otolaryngology—head and neck surgery: official journal of American Academy of Otolaryngology-Head and Neck Surgery. 2014; 151(4):612-8. Epub 2014/08/06. doi: 10.1177/0194599814545083. PubMed PMID: 25091190; PubMed Central PMCID: PMC4469264.
14. Felsen C N, Savariar E N, Whitney M, Tsien R Y. Detection and monitoring of localized matrix metalloproteinase upregulation in a murine model of asthma. American journal of physiology Lung cellular and molecular physiology. 2014; 306(8):L764-74. Epub 2014/02/11. doi: 10.1152/ajplung.00371.2013. PubMed PMID: 24508733; PubMed Central PMCID: PMC3989727.
15. Crisp J L, Savariar E N, Glasgow H L, Ellies L G, Whitney M A, Tsien R Y. Dual targeting of integrin alphavbeta3 and matrix metalloproteinase-2 for optical imaging of tumors and chemotherapeutic delivery. Molecular cancer therapeutics. 2014; 13(6):1514-25. Epub 2014/04/17. doi: 10.1158/1535-7163.MCT-13-1067. PubMed PMID: 24737028; PubMed Central PMCID: PMC4051287.
16. Adams S R, Yang H C, Savariar E N, Aguilera J, Crisp J L, Jones K A, et al. Anti-tubulin drugs conjugated to anti-ErbB antibodies selectively radiosensitize. Nature communications. 2016; 7:13019. Epub 2016/10/05. doi: 10.1038/ncomms13019. PubMed PMID: 27698471; PubMed Central PMCID: PMC5059467 findings described in this manuscript (S.R.A., E.N.S., R.Y.T. and S.J.A.). The remaining authors declare no competing financial interests.
17. Ferlay J, Shin H R, Bray F, Forman D, Mathers C, Parkin D M. Estimates of worldwide burden of cancer in 2008: GLOBOCAN 2008. International journal of cancer Journal international du cancer. 2010; 127(12):2893-917. doi: 10.1002/ijc.25516. PubMed PMID: 21351269.
18. Hinni M L, Ferlito A, Brandwein-Gensler M S, Takes R P, Silver C E, Westra W H, et al. Surgical margins in head and neck cancer: a contemporary review. Head & neck. 2013; 35(9):1362-70. Epub 2012/09/04. doi: 10.1002/hed.23110. PubMed PMID: 22941934.
19. Luryi A L, Chen M M, Mehra S, Roman S A, Sosa J A, Judson B L. Positive surgical margins in early stage oral cavity cancer: an analysis of 20,602 cases. Otolaryngology—head and neck surgery: official journal of American Academy of Otolaryngology-Head and Neck Surgery. 2014; 151(6):984-90. Epub 2014/09/27. doi: 10.1177/0194599814551718. PubMed PMID: 25257901.
20. Weissleder R, Mahmood U. Molecular imaging. Radiology. 2001; 219(2):316-33. Epub 2001/04/27. doi: 10.1148/radiology.219.2.r01ma19316. PubMed PMID: 11323453.
21. Egeblad M, Werb Z. New functions for the matrix metalloproteinases in cancer progression. Nature reviews Cancer. 2002; 2(3):161-74. Epub 2002/05/07. doi: 10.1038/nrc745. PubMed PMID: 11990853.
22. Bauvois B. New facets of matrix metalloproteinases MMP-2 and MMP-9 as cell surface transducers: outside-in signaling and relationship to tumor progression. Biochim Biophys Acta. 2012; 1825(1):29-36. Epub 2011/10/25. doi: 10.1016/j.bbcan.2011.10.001. PubMed PMID: 22020293.
23. Curino A, Patel V, Nielsen B S, Iskander A J, Ensley J F, Yoo G H, et al. Detection of plasminogen activators in oral cancer by laser capture microdissection combined with zymography. Oral oncology. 2004; 40(10):1026-32. Epub 2004/10/29. doi: 10.1016/j.oraloncology.2004.05.011. PubMed PMID: 15509494.
24. Coussens L M, Fingleton B, Matrisian L M. Matrix metalloproteinase inhibitors and cancer: trials and tribulations. Science. 2002; 295(5564):2387-92. Epub 2002/03/30. doi: 10.1126/science.1067100. PubMed PMID: 11923519.
25. Hamano Y, Zeisberg M, Sugimoto H, Lively J C, Maeshima Y, Yang C, et al. Physiological levels of tumstatin, a fragment of collagen IV alpha3 chain, are generated by MMP-9 proteolysis and suppress angiogenesis via alphaV beta3 integrin. Cancer Cell. 2003; 3(6):589-601. Epub 2003/07/05. PubMed PMID: 12842087; PubMed Central PMCID: PMCPMC2775452.
26. Montel V, Kleeman J, Agarwal D, Spinella D, Kawai K, Tarin D. Altered metastatic behavior of human breast cancer cells after experimental manipulation of matrix metalloproteinase 8 gene expression. Cancer research. 2004; 64(5):1687-94. Epub 2004/03/05. PubMed PMID: 14996728.
27. Somiari S B, Somiari R I, Heckman C M, Olsen C H, Jordan R M, Russell S J, et al. Circulating MMP2 and MMP9 in breast cancer—potential role in classification of patients into low risk, high risk, benign disease and breast cancer categories. Int J Cancer. 2006; 119(6):1403-11.
28. Oshima H, Nakayama M, Han T S, Naoi K, Ju X, Maeda Y, et al. Suppressing TGFbeta Signaling in Regenerating Epithelia in an Inflammatory Microenvironment Is Sufficient to Cause Invasive Intestinal Cancer. Cancer Res. 2015; 75(4):766-76.
29. Willis A L, Sabeh F, Li X Y, Weiss S J. Extracellular matrix determinants and the regulation of cancer cell invasion stratagems. Journal of Microscopy. 2013; 251(3):250-60. doi: 10.1111/jmi.12064.
30. Perentes J Y, Kirkpatrick N D, Nagano S, Smith E Y, Shaver C M, Sgroi D, et al. Cancer cell-associated MT1-MMP promotes blood vessel invasion and distant metastasis in triple-negative mammary tumors. Cancer research. 2011; 71(13):4527-38. Epub 2011/05/17. doi: 10.1158/0008-5472.can-10-4376. PubMed PMID: 21571860.
31. Uloza V, Liutkevicius V, Pangonyte D, Saferis V, Lesauskaite V. Expression of matrix metalloproteinases (MMP-2 and MMP-9) in recurrent respiratory papillomas and laryngeal carcinoma: clinical and morphological parallels. Eur Arch Otorhinolaryngol. 2011; 268(6):871-8. Epub 2011/01/25. doi: 10.1007/s00405-011-1494-1. PubMed PMID: 21259063.
32. Wittekindt C, Jovanovic N, Guntinas-Lichius O. Expression of matrix metalloproteinase-9 (MMP-9) and blood vessel density in laryngeal squamous cell carcinomas. Acta Otolaryngol. 2011; 131(1):101-6. Epub 2010/09/30. doi: 10.3109/00016489.2010.506886. PubMed PMID: 20873997.
33. Liu W W, Zeng Z Y, Wu Q L, Hou J H, Chen Y Y. Overexpression of MMP-2 in laryngeal squamous cell carcinoma: a potential indicator for poor prognosis. Otolaryngology—head and neck surgery: official journal of American Academy of Otolaryngology-Head and Neck Surgery. 2005; 132(3):395-400. Epub 2005/03/05. doi: 10.1016/j.otohns.2004.09.050. PubMed PMID: 15746850.
34. Mallis A, Teymoortash A, Mastronikolis N S, Werner J A, Papadas T A. MMP-2 expression in 102 patients with glottic laryngeal cancer. Eur Arch Otorhinolaryngol. 2012; 269(2):639-42. Epub 2011/06/15. doi: 10.1007/s00405-011-1625-8. PubMed PMID: 21667117.
35. Zhou C X, Gao Y, Johnson N W, Gao J. Immunoexpression of matrix metalloproteinase-2 and matrix metalloproteinase-9 in the metastasis of squamous cell carcinoma of the human tongue. Aust Dent J. 2010; 55(4):385-9. Epub 2010/12/24. doi: 10.1111/j.1834-7819.2010.01258.x. PubMed PMID: 21174909.
36. Kwaan H C, Mazar A P, McMahon B J. The apparent uPA/PAI-1 paradox in cancer: more than meets the eye. Semin Thromb Hemost. 2013; 39(4):382-91. Epub 2013/03/28. doi: 10.1055/s-0033-1338127. PubMed PMID: 23532574.
37. Savariar E N, Felsen C N, Nashi N, Jiang T, Ellies L G, Steinbach P, et al. Real-time in vivo molecular detection of primary tumors and metastases with ratiometric activatable cell-penetrating peptides. Cancer research. 2013; 73(2):855-64. Epub 2012/11/29. doi: 10.1158/0008-5472.CAN-12-2969. PubMed PMID: 23188503; PubMed Central PMCID: PMC3799878.
38. Duspara, Petar A., et al. “Synthesis and reactivity of N-alkyl carbamoylimidazoles: development of N-methyl carbamoylimidazole as a methyl isocyanate equivalent.” The Journal of organic chemistry 77.22 (2012): 10362-10368.
39. Braun, Alexandra C., et al. “Bioorthogonal strategies for site-directed decoration of biomaterials with therapeutic proteins.” Journal of Controlled Release (2018).
40. Saxon, Eliana, Joshua I. Armstrong, and Carolyn R. Bertozzi. “A “traceless” Staudinger ligation for the chemoselective synthesis of amide bonds.” Organic letters 2.14 (2000): 2141-2143.
41. Jain, Nareshkumar, et al. “Current ADC linker chemistry.” Pharmaceutical research 32.11 (2015): 3526-3540.
42. Current A D. Linker Chemistry Jain, Nareshkumar; Smith, Sean W.; Ghone, Sanjeevani; Tomczuk, Bruce. Pharmaceutical Research. 2015; 32(11):3526-40.
43. Denkbas E B, Ottenbrite R M. Perspectives on: chitosan drug delivery systems based on their geometries. Journal of Bioactive and Compatible polymers. 2006 July;21(4):351-68
44. Richard, Jean-Alexandre, et al. “Latent fluorophores based on a self-immolative linker strategy and suitable for protease sensing.” Bioconjugate chemistry 19.8 (2008): 1707-1718.
45. Hermanson, Greg T. Bioconjugate techniques. Academic press, 2013.
46. Whiteoak, Christopher J., et al, “A powerful aluminum catalyst for the synthesis of highly functional organic carbonates.” Journal of the American Chemical Society 135.4 (2013): 1228-1231.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

All publications, including patent documents and scientific articles, referred to in this application and the bibliography and attachments are incorporated by reference in their entirety for all purposes to the same extent as if each individual publication were individually incorporated by reference.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An activatable specific binding member complex, comprising:
a) a first specific binding member, comprising:
1) a first specific binding region, with binding affinity for a first target binding domain;
2) a first linker site; and
b) a complementary binding member/linker according to the following formula:
R¹—R²—(R³)ₘ—R⁴—(R⁵)ₙ—R⁶
wherein R¹ is substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl; R² is a first complementary binding member; R³ is a first sublinker; m is either 0 or 1; R⁴ is a cleavable substrate; R⁵ is a second sublinker; n is either 0 or 1; R⁶ is the attachment point to the first linker site of the first specific binding member.

2. The activatable specific binding member complex of claim 1, wherein said linker allows specific binding and reversible binding of said first specific binding region with said first complementary binding member.

3. The activatable specific binding member complex of claim 1, wherein when said linker is cleaved at said cleavable substrate, said first specific binding member and said first complementary binding member become capable of dissociating from each other.

4. The activatable specific binding member complex of claim 1, wherein when said linker is cleaved at said cleavable substrate, said first specific binding region and said first complementary binding member dissociate from each other.

5. The activatable specific binding member complex of claim 4, wherein when said linker is cleaved at said cleavable substrate, said first specific binding region and said first complementary binding member dissociate from each other, thus forming an activated specific binding member; and wherein said activated specific binding member functions such that said first specific binding region can bind with at least one moiety other than said first complementary binding member.

6. The activatable specific binding member complex of claim 5, wherein the at least one moiety other than said first complementary binding member is a first target binding domain.

7. The activatable specific binding member complex of claim 1, wherein said first specific binding member comprises between about 25% and about 99% of a CDR of an antibody selected from the group consisting of adalimumab, bezlotoxumab, avelumab, dupilumab, durvalumab, brodalumab, reslizumab, olaratumab, daratumumab, elotuzumab, necitumumab, infliximab, obiltoxaximab, atezolizumab, secukinumab, mepolizumab, nivolumab, alirocumab, idarucizumab, evolocumab, dinutuximab, bevacizumab, pembrolizumab, ramucirumab, vedolizumab, siltuximab, alemtuzumab, trastuzumab emtansine, pertuzumab, infliximab, obinutuzumab, brentuximab, raxibacumab, belimumab, ipilimumab, denosumab, ofatumumab, besilesomab, tocilizumab, canakinumab, golimumab, ustekinumab, certolizumab pegol, catumaxomab, eculizumab, ranibizumab, panitumumab, natalizumab, bevacizumab, omalizumab, cetuximab, efalizumab, ibritumomab tiuxetan, fanolesomab, adalimumab, tositumomab, iodine 131 tositumomab, alemtuzumab, trastuzumab, gemtuzumab ozogamicin, infliximab, palivizumab, necitumumab, basiliximab, rituximab, votumumab, sulesomab, arcitumomab, imiciromab, capromab, nofetumomab, and abciximab.

8. The activatable specific binding member complex of claim 1, wherein said first specific binding member comprises between about 25% and about 99% of a CDR of an antibody selected from the group consisting of cetuximab, trastuzumab, or adalimumab.

9. The activatable specific binding member complex of claim 1, wherein said first linker site is a lysine or a cysteine.

10. The activatable specific binding member complex of claim 1, wherein said R⁴ comprises an uPA cleavage substrate, an MMP cleavage substrate, or a thrombin cleavage substrate.

11. The activatable specific binding member complex of claim 1, wherein when m is 1, R³ comprises a member selected from the group consisting of PEG, a protein nucleic acid (PNA), a D amino acid, an L amino acid, a lipophilic residue, an SPDB disulfide, MCC (maleimidomethyl cyclohexane-1-carboxylate), sulfo-SPDB which adds a charged polar group, hydrazine, and combinations thereof.

12. The activatable specific binding member complex of claim 1, wherein when m is 1, R³ is PEG.

13. The activatable specific binding member complex of claim 1, wherein when n is 1, R⁵ comprises a member selected from the group consisting of PEG, a protein nucleic acid (PNA), a D amino acid, an L amino acid, a lipophilic residue, an SPDB disulfide, MCC (maleimidomethyl cyclohexane-1-carboxylate), sulfo-SPDB which adds a charged polar group, hydrazine, and combinations thereof.

14. The activatable specific binding member complex of claim 1, wherein when n is 1, R⁵ is PEG.

15. The activatable specific binding member complex of claim 1, wherein said first target binding domain comprises a member selected from the group consisting of EGFR, HER-2, VEGF, CD20, CTLA-1 PDL-1, C. difficile toxin B, TNFα, PD-L1, IL-4Ra, CD20, IL-17RA, IL-5, PDGFR-α, D38, SLAMF7, EGFR, PA component of B. anthracis toxin, interleukin-17A, IL-5, PD-1, PCSK9, dabigatran etexilate, LDL-C/PCSK9, GD2, CD19, VEGF, Integrin-α4β7, cCLB8, CD52, HER2, CD30, Bacillus anthracis protective antigen, BLyS, CTLA-4, RANKL, NCA-95, IL-6 receptor, IL-1B, IL-12/IL-23, EpCAM and CD3, Complement C5, VLA-4, EpCAM, IgE, CD11a, CD15, CD33, F-protein of RS virus, CD25 (a chain of IL2 receptor), Cytokeratintumor-associated antigen, Human cardiac myosin, NCA90, Human CEA (carcinoembryonic antigen), Tumor surface antigen PSMA, Carcinoma-associated antigen GPIIb/IIIa, integrins, an antibody drug target, any cell determinant, or a combination.

16. The activatable specific binding member complex of claim 1, wherein said first target binding domain comprises a member selected from the group consisting of EGFR, HER-2, and TNFα.

17. The activatable specific binding member complex of claim 1, wherein said first specific binding region comprises between about 25% and about 99% of a CDR of cetuximab, said first target binding domain is EGFR, said first linker site is lysine or cysteine.

18. A composition comprising activatable specific binding member complexes, comprising:
a) a first specific binding member, comprising:
1) a first specific binding region, with binding affinity for a first target binding domain;
2) a first linker site which is a lysine; and
b) a complementary binding member/linker according to the following formula:
R¹—R²—(R³)ₘ—R⁴—(R⁵)ₙ—R⁶
wherein R¹ is substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl; R² is a first complementary binding member; R³ is a first sublinker; m is either 0 or 1; R⁴ is a cleavable substrate; R⁵ is a second sublinker; n is either 0 or 1; R⁶ is the attachment point to the first linker site of the first specific binding member.

19. A composition comprising activatable specific binding member complexes, comprising:
a) a first specific binding member, comprising:
1) a first specific binding region, with binding affinity for a first target binding domain;
2) a first linker site which is a cysteine; and
b) a complementary binding member/linker according to the following formula:
R¹—R²—(R³)ₘ—R⁴—(R⁵)ₙ—R⁶
wherein R¹ is substituted or unsubstituted alkyl, substituted or unsubstituted succinyl, substituted or unsubstituted acryl, substituted or unsubstituted benzoyl, substituted or unsubstituted alkyl ester, or substituted or unsubstituted alkyl carbonyl; R² is a first complementary binding member; R³ is a first sublinker; m is either 0 or 1; R⁴ is a cleavable substrate; R⁵ is a second sublinker; n is either 0 or 1; R⁶ is the attachment point to the first linker site of the first specific binding member.

20. A composition comprising activatable specific binding member complexes, prepared by a process described herein.
